(19) United States
(12) Patent Application Publication
(10) Pub. No.: US 2023/0000889 A1
(43) Pub. Date: Jan. 5, 2023

(54) METHODS OF TREATING ACUTE MYELOID LEUKEMIA AND MANAGING CYTOPENIA

(71) Applicants: GENENTECH, INC., South San Francisco, CA (US); ABBVIE INC., North Chicago, IL (US)

(72) Inventors: Ahmed Hamed SALEM, Libertyville, IL (US); Jalaja POTLURI, North Chicago, IL (US); Jiuhong ZHA, Chicago, IL (US); Mehrdad MOBASHER, San Francisco, CA (US); Wan-Jen HONG, Mountain View, CA (US)

(73) Assignees: GENENTECH, INC., South San Francisco, CA (US); ABBVIE INC., North Chicago, IL (US)

(21) Appl. No.: 17/834,642

(22) Filed: Jun. 7, 2022

Related U.S. Application Data
(60) Provisional application No. 63/208,449, filed on Jun. 8, 2021.

(51) Int. Cl. A61K 31/706; A61K 31/635; A61K 9/00; A61P 35/02

(52) U.S. Cl. CPC A61K 31/706; A61K 31/635; A61K 9/0053; A61K 9/0019; A61P 35/02

(57) ABSTRACT

Provided herein are methods for treating acute myeloid leukemia (AML) in a patient with cytopenia when on a combination therapy of venetoclax and azacitidine. Also provided herein are methods of managing a cytopenia in such patient.

### 1. CROSS REFERENCE

This application claims the benefit of U.S. Provisional Application No. 63/208,449, filed Jun. 8, 2021, the disclosure of which is incorporated by reference herein in its entirety.

### 2. FIELD

Disclosed herein are methods for treating acute myeloid leukemia (AML) in a patient with cytopenia when on a combination therapy of venetoclax and azacitidine. Also provided herein are methods of managing cytopenia in such a patient.

### 3. BACKGROUND

Hematologic malignancies are highly dependent upon the anti-apoptotic protein Bcl-2 for survival. Over-expression of Bcl-2 is associated with tumor initiation, disease progression, and drug resistance, and is thus a compelling target for anti-tumor therapy. Venetoclax is a potent, selective and orally bioavailable small molecule inhibitor of Bcl-2 that binds with >1,000-fold higher affinity for Bcl-2<0.010 nM) than for Bcl-X_{L}(Kᵢ=48 nm) or Mcl-1(Kᵢ>444 nM). See, e.g., Souers et al., Nat Med. 2013; 19(2):202-8 (“Souers et al., 2013”). In vitro, venetoclax has demonstrated cell killing activity against patient-derived chronic lymphocytic leukemia (CLL) cells and a variety of lymphoma and leukemia cell lines, including acute myeloid leukemia (AML). See, e.g., Souers et al., 2013.

Bcl-2 over-expression has also been implicated in the maintenance and survival of AML cells and has been associated with resistance to chemotherapeutics. In addition, high levels of Bcl-2 are associated with poor survival in a subset of patients with this disease. See, e.g., Konopleva et al., Cancer Cell. 2006; 10(5):375-88; see also Tsao et al., Ann Hematol. 2012; 91(12):1861-70. Venetoclax has also demonstrated killing of AML leukemic stem/progenitor cells ex vivo and antitumor efficacy in vivo, inhibiting the growth of AML cell lines or AML patient-derived primary cells systemically engrafted into immunocompromised mice. See, e.g., Pan et al., Cancer Discov. 2014; 4(3):362-75. Single agent venetoclax is was previously studied in relapsed/refractory (R/R) AML and was found to induce rapid reduction in blast counts in some patients indicating activity in this disease. See Konopleva et al., Cancer Discov. 2016; 6(10): 1106-17. However, not all AML cell lines, primary patient samples, or patients treated with single agent venetoclax were found to be sensitive, and there is biologic rationale for combining venetoclax with certain chemotherapeutic agents in the treatment of AML.

Treatments for AML are sought in view that certain patients respond poorly or develop resistance to standard induction therapy with chemotherapeutic agents such as cytarabine. Combinations of venetoclax and chemotherapeutic agents and hypomethylating agents commonly used in the treatment of AML have been tested. For example, venetoclax was administered daily during the 28-day cycles in combination with decitabine (20 mg/m² intravenously on Days 1-5 once every 28 days) or azacitidine (75 mg/m² intravenously or subcutaneously on Days 1-7 once every 28 days). Preliminary safety and efficacy data are available from a phase Ib dose escalation and expansion of this trial. See DiNardo et al., “A phase 1b study of venetoclax (ABT-199/GDC-0199) in combination with decitabine or azacitidine in treatment-naive patients with acute myelogenous leukemia who are >65 years and not eligible for standard induction therapy,” presented at: 57th Annual Meeting of the American Society of Hematology; Dec. 5-8, 2015; Orlando, Fla. Abstract 327. See also DiNardo et al., Lancet Oncol. 2018; 19(2):216-28; and DiNardo et al., Blood. 2019; 133(1):7-17. Despite advantages such as, e.g., improved response demonstrated with combination therapy, as compared to response rates in monotherapy response rates, occurrence of cytopenia, such as, e.g., neutropenia and thrombocytopenia, are often observed in many patients undergoing such treatment, requiring further inventions.

Cytopenias, including anemia, leukopenia, neutropenia, and thrombocytopenia, are common in subjects with AML. Subjects with baseline neutropenias or those with secondary AML might be particularly at high risk for developing serious cytopenias. There is an unmet need for new protocols that manage cytopenia in patients on combination therapies of venetoclax and azacitidine to treat AML.

### 4. SUMMARY

A subpopulation of patients being treated for acute myeloid leukemia (AML) experience cytopenia, such as neutropenia or thrombocytopenia, including after having achieved remission of the AML. The present disclosure provides methods for treating acute myeloid leukemia (AML) in a patient with a cytopenia when on a combination therapy of venetoclax and azacitidine. The present disclosure also provides methods of managing a cytopenia in such a patient, including treating cycle delays, treatment interruptions, and dosage reductions. The present disclosure also provides methods of managing a cytopenia in such a patient without negatively impacting treatment outcomes, such as overall survival (OS) efficacy outcome, in said patient. The present disclosure further provides methods of managing a cytopenia in such a patient and positively impacting treatment outcomes, such as overall survival (OS) efficacy outcome, for example improving treatment outcomes, such as improving or extending overall survival (OS) efficacy outcome, in said patient.

Accordingly, in a first aspect, provided herein is a method of treating acute myeloid leukemia (AML) in a patient with neutropenia and/or thrombocytopenia when on a combination therapy of venetoclax and azacitidine, the method comprising:
a) administering venetoclax and azacitidine to a patient in need thereof for one or more 28-day cycles of a multiple 28-day cycle dosing regimen, wherein each 28-day cycle of the one or more 28-day cycles comprises orally administering 400 mg venetoclax on days 1-28 of the 28-day cycle, and administering azacitidine 75 mg/m² intravenously or subcutaneously on days 1-7 of the 28-day cycle; and
b) interrupting the administration of venetoclax and azacitidine to the patient after a first 28-day cycle of the one or more 28-day cycles in step (a), upon the patient having:
   (i) an occurrence of neutropenia, until the patient has absolute neutrophil count (“ANC”) greater than or equal to 500 cells/μL within 14 days from the day of the interruption, and/or
   (ii) an occurrence of thrombocytopenia, until the patient has a platelet count greater than or equal to 50,000 cells/μL within 14 days from the day of the interruption; and resuming the administration of venetoclax and azacitidine for the next 28-day cycle of the one or more 28-day cycles in step (a), wherein the resumed administration of venetoclax and azacitidine starts on the same day.

In a second aspect, provided herein is a method of managing cytopenia in a patient with neutropenia and/or thrombocytopenia when on a combination therapy of venetoclax and azacitidine to treat acute myeloid leukemia (AML), the method comprising:
a) administering venetoclax and azacitidine to a patient in need thereof for one or more 28-day cycles of a multiple 28-day cycle dosing regimen, wherein each 28-day cycle of the one or more 28-day cycles comprises orally administering 400 mg venetoclax on days 1-28 of the 28-day cycle, and administering azacitidine 75 mg/m² intravenously or subcutaneously on days 1-7 of the 28-day cycle; and
b) interrupting the administration of venetoclax and azacitidine to the patient after a first 28-day cycle of the one or more 28-day cycles in step (a), upon the patient having:
   (i) an occurrence of neutropenia, until the patient has absolute neutrophil count (“ANC”) greater than or equal to 500 cells/μL within 14 days from the day of the interruption, and/or
   (ii) an occurrence of thrombocytopenia, until the patient has a platelet count greater than or equal to 50,000 cells/μL within 14 days from the day of the interruption; and resuming the administration of venetoclax and azacitidine for the next 28-day cycle of the one or more 28-day cycles in step (a), wherein the resumed administration of venetoclax and azacitidine starts on the same day.

Other aspects and embodiments will be evident to those skilled in the art upon reading the present specification.

### 5. BRIEF DESCRIPTION OF DRAWINGS

FIG. 1 shows various dosing modifications that may be implemented to manage a cytopenia in a patient when on a combination therapy of venetoclax and azacitidine.

FIGS. 2A and 2B shows exemplary dose interruptions following a Grade 4 cytopenia (e.g., neutropenia and/or thrombocytopenia) event during a 28-day cycle, and resumption of the administration of venetoclax and azacitidine on the same day for the next 28-day cycle.

FIG. 3 shows an exemplary study design for a double-blind, placebo-controlled, multicenter phase 3 study of venetoclax and azacitidine in patients with acute myeloid leukemia (AML).

FIG. 4 shows an exemplary dosing modification for venetoclax and azacitidine to manage a cytopenia (e.g., neutropenia and/or thrombocytopenia).

FIG. 5 shows an exemplary dosing modification for venetoclax and azacitidine to manage a cytopenia (e.g., neutropenia and/or thrombocytopenia).

FIG. 6 shows an exemplary dosing modification for venetoclax and azacitidine to manage a cytopenia (e.g., neutropenia and/or thrombocytopenia).

FIG. 7 shows an exemplary dosing modification for venetoclax and azacitidine to manage a cytopenia (e.g., neutropenia and/or thrombocytopenia).

FIGS. 8A and 8B show overall survival in patients with CR/CRh with post-remission cytopenia. FIG. 8A shows patients who switched to 21-day dosing following their first post-remission cytopenia. FIG. 8B shows patients who remained on 28-day dosing and reduced venetoclax dosing duration in later cycles.

### 6. DETAILED DESCRIPTION

### 6.1 Definitions

Unless defined otherwise, technical and scientific terms used herein have the same meaning as commonly understood by those skilled in the art to which the present disclosure belongs.

As used herein, the term “venetoclax” means “4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin5-yloxy)benzamide),” which is described in International Publication No. WO2010/138588 and in US publication No. US2010/0305122, both of which are incorporated by reference herein. The chemical structure for venetoclax is shown below:

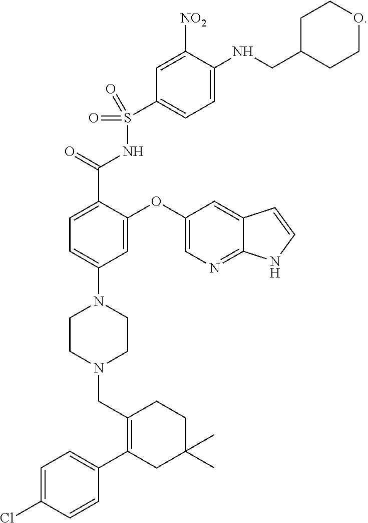 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

As used herein, the term “azacitidine” means “4-amino-1-β-D-ribofuranosyl-s-triazin-2(1H)-one” (also known as “4-amino-1-((2R,3R,4S,5R)-3,4-dihydroxy-5-(hydroxymethyl)tetrahydrofuran-2-yl)-1,3,5-triazin-2(1H)-one”), for which the chemical structure is shown below:

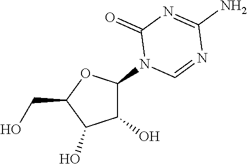 [SMILES: Nc1ncn([C@@H]2O[C@H](CO)[C@@H](O)[C@H]2O)c(=O)n1]

### 4-amino-1-((2R,3R,4S,5R)-3,4-dihydroxy-5-(hydroxymethyl)tetrahydrofuran-2-yl)-1,3,5-triazin-2(1H)-one

As used herein, the term “cytopenia” means a deficiency in the number of blood cells and/or a deficiency in the number of a given type of blood cell. Cytopenia includes anemia, leukopenia, neutropenia, thrombocytopenia, and pancytopenia.

As used herein, the term “neutropenia” means a deficiency in the number of circulating neutrophils, for example, an absolute neutrophil count (ANC) less than 1,500 cells/μl (Grades 2 or higher). As used herein, the following grades of neutropenia are: Grade 0: ANC≥2,000 cells/μl; Grade 1: ANC≥1,500 cells/μl to <2,000 cells/μl; Grade 2: ANC≥1,000 cells/μl to <1,500 cells/μl; Grade 3: ANC≥500 cells/μl to <1,000/μl; Grade 4: ANC≤500 cells/μl. See, e.g., Common Terminology Criteria for Adverse Events (CTCAE), Version 5.0, November 2017, National Institutes of Health, National Cancer Institute, incorporated herein by reference in its entirety. It will be understood by those skilled in the art that Grade 0 neutropenia indicates the absence of neutropenia, that is, where there is no deficiency in the number of circulating neutrophils.

As used herein, the term “thrombocytopenia” means a deficiency in the number of circulating platelets, for example, a platelet count less than 100,000 cells/μl. As used herein, the following grades of thrombocytopenia are: Grade 0: platelet count≥150,000 cells/μl; Grade 1: platelet count≥75,000 cells/μl to <150,000 cells/μl; Grade 2: platelet count≥50,000 cells/μl to <75,000 cells/μl; Grade 3: platelet count≥25,000 cells/μl to <50,000/μl; Grade 4: platelet count≤25,000 cells/μl. See, e.g., Common Terminology Criteria for Adverse Events (CTCAE), Version 5.0, November 2017, National Institutes of Health, National Cancer Institute. Grade 1 thrombocytopenia may also be categorized as Grade 1A: platelet count≥100,000 cells/μl to <150,000 cells/μl; and Grade 1B: platelet count≥75,000 cells/μl to <100,000 cells/μl. It will be understood by those skilled in the art that Grade 0 thrombocytopenia indicates the absence of thrombocytopenia, that is, where there is no deficiency in the number of circulating platelets.

As used herein, the term “absolute neutrophil count (ANC)” means the total number of neutrophils in the white blood cell count. Those skilled in the art will know how to calculate ANC. As an example, ANC may be calculated as follows: ANC=WBC (cells/4)×[percent (PMNs+bands)÷100]; where “WBC” is the white blood cell count, “PMNs” is the percent of neutrophils, and “bands” are young neutrophils.

As used herein, the term “blast clearance” means bone marrow with <5% bone marrow blasts (myeloblasts).

As used herein, the term “complete remission (CR)” means bone marrow with <5% myeloblasts (blasts), the absence of circulating blasts and blasts with Auer rods, hematologic recovery (as evidenced by ANC≥1,000 cells/4, and platelet count≥100,000 cells/4), with no need for red blood cell (RBC) transfusions, and the absence of extramedullary disease.

As used herein, the term “complete remission with partial hematological recovery (CRh)” (also referred to as “complete remission with partial count recovery (CRh)”) means all of the criteria for CR except for partial recovery of peripheral blood counts (as evidenced by ANC≥500 cells/μL and platelet count≥50,000 cells/μl).

As used herein, the term “complete remission with incomplete marrow recovery (CRi)” means all of the criteria for CR except for residual cytopenia (as evidenced by ANC≤1,000 cells/μL or platelet count<100,000 cells/μL).

As used herein, the term “post-remission cytopenia” or “post-remission cytopenia event” means a Grade 4 neutropenia event or a Grade 4 thrombocytopenia event lasting>7 days in a patient who had achieved complete remission (CR) or complete remission with partial hematological recovery (CRh).

As used herein, the term “morphologic leukemia-free state (MLFS)” means less than 5% blasts in an aspirate sample with marrow spicules and with a count of at least 200 nucleated cells, absence of circulating blasts and extramedullary disease without peripheral blood count recovery that meet the thresholds for either CR or CRi.

As used herein, the term “treating” means reversing, alleviating, inhibiting the progress of, or preventing, either partially or completely, a cancer in a patient, for example, acute myeloid leukemia (AML) in a patient. For example, in certain embodiments, treating means reducing the number of circulating blasts, e.g., to less than 5%. In other certain embodiments, treating means the patient has achieved one or more of a complete recovery (CR), complete remission with partial hematological recovery (CRh), complete remission with incomplete count recovery (CRi), and a morphologic leukemia-free state (MLFS). In certain embodiments, “treating” means increasing the survival of an AML patient.

As used herein, the term “a method of treating” or its equivalent, when applied to, for example, cancer refers to a procedure or course of action that is designed to reduce or eliminate the number of cancer cells in a patient, or to alleviate the symptoms of a cancer. “A method of treating” cancer, for example, acute myeloid leukemia (AML), does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a patient, is nevertheless deemed to induce an overall beneficial course of action.

As used herein, “interrupt,” “interrupting,” “interruption,” and the like, in the context of, for example, interrupting the administration of venetoclax and/or azacitidine, mean a break, disruption, and/or delay in the administration of venetoclax and/or azacitidine. As such, terms such as interrupt, break, disrupt, delay; interrupting, breaking, disrupting, delaying; or interruption, disruption, delay, etc., may be used interchangeably.

As used herein, the term “patient” typically refers to a human in need of treating a cancer, for example, acute myeloid leukemia (AML). Such patient may exhibit a cytopenia, for example, neutropenia and/or thrombocytopenia, because of AML or as a result of the treatment for AML. However, the term “patient” can also refer to non-human animals, preferably mammals such as dogs, cats, horses, cows, pigs, sheep and non-human primates, among others.

### 6.2 Methods of Treating Aml and/or Managing Cytopenia

In one aspect, provided herein is a method of treating a cancer, for example, acute myeloid leukemia (AML), in a patient with cytopenia when on a combination therapy of venetoclax and azacitidine. Without being bound by any theory or mechanism, it is believed that an AML patient who develops a cytopenia when on combination therapy can be treated with certain regimens and doses of the active agents to reduce the cytopenia and continue with combination therapy to treat AML with a reduced likelihood of re-occurrence of the cytopenia.

In certain embodiments, a method provided herein comprises interrupting administration of venetoclax and azacitidine to a patient in need thereof after a first cycle of one or more administration cycles, upon the patient having an occurrence of cytopenia (after patient has achieved remission, where the occurrence of cytopenia is not caused by a relapse of the AML or due to underlying cancer), until the patient recovers from the cytopenia, and resuming the administration of venetoclax and azacitidine for the next cycle of the one or more cycles, wherein the resumed administration of venetoclax and azacitidine starts on the same day.

It will be understood that in AML patients, a cause of cytopenia, e.g., neutropenia or thrombocytopenia, can be the disease itself as opposed to the treatment of the disease. For example, the expansion of one clone of white blood cells can prevent other white cells from flourishing and the diversity of those neutrophils, not necessarily their total number, would be reduced. As such, in certain embodiments of the methods provided herein, the cytopenia, e.g., neutropenia or thrombocytopenia, is not related to or caused by AML, the underlying disease. In some embodiments, the cytopenia, e.g., neutropenia or thrombocytopenia, in the treated patient occurs wherein the bone marrow of the patient has less than 5% blasts, or wherein there is an absence of circulating blasts and/or blasts with Auer rods. In some embodiments, the cytopenia, e.g., neutropenia or thrombocytopenia, in the treated patient occurs wherein the bone marrow has less than 5% blasts. In some embodiments, the occurrence of cytopenia includes an occurrence of neutropenia, thrombocytopenia, or both.

In certain embodiments, the patient exhibits complete remission with incomplete count recovery (CRi) at one or more steps of the methods described herein.

In certain embodiments, the patient exhibits a morphologic leukemia-free state (MLFS) at one or more steps of the methods described herein.

In certain embodiments, the venetoclax and azacitidine are administered for one or more 28-day cycles of a multiple 28-day cycle dosing regimen, prior to the interruption.

In certain embodiments, each 28-day cycle of the one or more 28-day cycles comprises orally administering 400 mg venetoclax on days 1-28 of the 28-day cycle, and administering azacitidine 75 mg/m² intravenously or subcutaneously on 7 days of the 28-day cycle. In certain embodiments, azacitidine is administered on 7 consecutive days of the 28-day cycle. In certain embodiments, azacitidine is administered on 7 nonconsecutive days of the 28-day cycle. In certain embodiments, azacitidine is administered in a combination of consecutive and nonconsecutive days of the 28-day cycle, so long as the total number of days azacitidine is administered is 7 days. In certain embodiments, each 28-day cycle of the one or more 28-day cycles comprises orally administering 400 mg venetoclax on days 1-28 of the 28-day cycle, and administering azacitidine 75 mg/m² intravenously or subcutaneously on days 1-5 and days 8-9 of the 28-day cycle. In certain embodiments, each 28-day cycle of the one or more 28-day cycles comprises orally administering 400 mg venetoclax on days 1-28 of the 28-day cycle, and administering azacitidine 75 mg/m² intravenously or subcutaneously on days 1-7 days of the 28-day cycle.

In certain embodiments, prior to interruption of the administration of venetoclax and azacitidine, the patient has achieved one or more of a complete recovery (CR), complete remission with incomplete count recovery (CRi), complete remission with partial hematological recovery (CRh), and a morphologic leukemia-free state (MLFS), as described herein.

Bone marrow blast levels may be determined by any present or future methodology known in the art to those skilled in the art.

In certain embodiments, a bone marrow assessment (e.g., bone marrow aspirate), biopsy, and blood count is performed on the patient to determine CR, CRi, CRh, and/or MLFS.

In certain embodiments, if the patient does not recover from the cytopenia following interruption of the administration of venetoclax and azacitidine, and the cytopenia is not caused by a relapse of AML, the patient is excluded from the method of treating AML.

In certain embodiments, if the patient does not recover from the cytopenia following interruption of the administration of venetoclax and azacitidine, the patient enters discussion with appropriate medical personnel to determine whether resumption or treatment or other therapeutic steps are necessary.

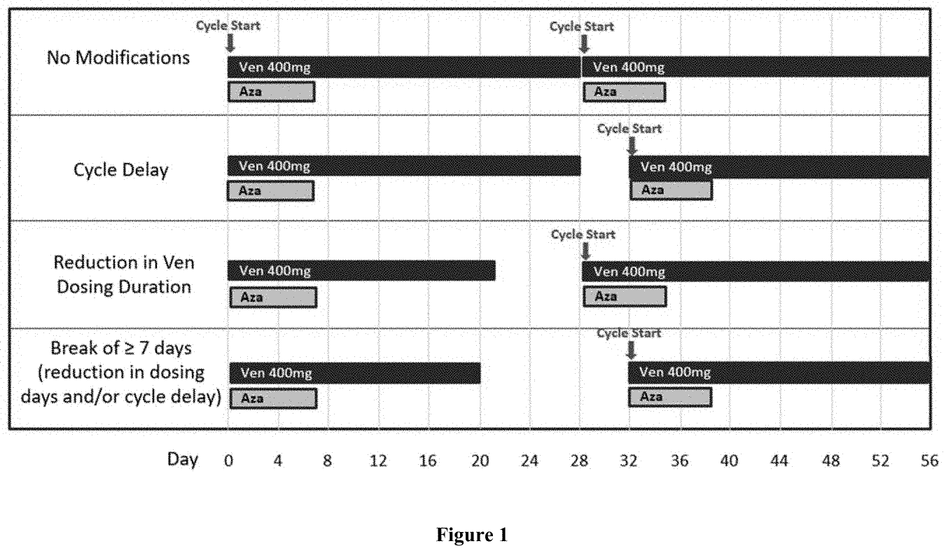

Disease response may be assessed via bone marrow aspirate, biopsy and blood count at the end of the first cycle of the multiple 28-day cycle dosing regimen, and at least every 3 cycles thereafter. After patients achieve blast clearance (bone marrow blasts<5%), various dosing modifications may be implemented to manage cytopenia, as shown in FIG. 1. For example, if during or at the end of a first cycle, incomplete count recovery is assessed (e.g., CRi or MLFS), or cytopenia observed, the upcoming cycle may be delayed as follows: (i) venetoclax is interrupted from Day 29 until ANC reaches a certain threshold, e.g., ≥1,500 cells/μL, ≥1,000 cells/μL, or ≥500 cells/μL, or platelet count reaches a certain threshold, e.g., ≥100,000 cells/μL, ≥75,000 cells/μL, ≥50,000 cells/μL, or ≥25,000 cells/μL, or up to 14 days (if no recovery by Day 42, the patient should enter discussion with appropriate medical personnel to determine whether exclusion from the treatment, resumption of treatment, and/or other therapeutic steps are necessary); (ii) azacitidine is also interrupted from Day 29 until ANC reaches a certain threshold, e.g., ≥1,500 cells/μL, ≥1,000 cells/μL, or ≥500 cells/μL, or platelet count reaches a certain threshold, e.g., ≥100,000 cells/μL, ≥75,000 cells/μL, ≥50,000 cells/μL, or ≥25,000 cells/μL, or up to 14 days; and (iii) assuming recovery by Day 42, venetoclax and azacitidine are resumed on the same day after the interruption. If during or at the end of a second cycle, a new cytopenia is observed (after prior ANC recovery and/or platelet count recovery) lasting more than one week, unless due to underlying disease, e.g., relapse, the upcoming cycle may be delayed as follows: venetoclax is interrupted once cycle completed and until ANC reaches a certain threshold, e.g., ≥1,500 cells/μL, ≥1,000 cells/μL, or ≥500 cells/μL, or platelet count reaches a certain threshold, e.g., ≥100,000 cells/μL, ≥75,000 cells/μL, ≥50,000 cells/μL, or ≥25,000 cells/μL, or up to 14 days, unless medically necessary to interrupt drug within cycle (if no recovery by Day 42, the patient should enter discussion with appropriate medical personnel to determine whether exclusion from the treatment, resumption of treatment, and/or other therapeutic steps, e.g., adjusted azacitidine dose are necessary). If during or at the end of a third cycle, a subsequent cytopenia is observed lasting more than one week (after prior ANC recovery and/or platelet count recovery), the upcoming cycle may be reduced in duration as follows: (i) the next treatment cycle is delayed until ANC reaches a certain threshold, e.g., ≥1,500 cells/4, ≥1,000 cells/4, or ≥500 cells/4, or platelet count reaches a certain threshold, e.g., ≥100,000 cells/4, ≥75,000 cells/μL, ≥50,000 cells/4, or ≥25,000 cells/4, or up to 14 days; and (ii) venetoclax is administered for 21/28 days for subsequent cycles. If during or at the end of a fourth cycle, a 25% increase from the nadir (e.g., the lowest ANC and/or platelet count measured during the multiple 28-day cycle dosing regimen) is not achieved within 14 days after completion of the cycle and/or if recovery after the third or fourth cycles takes more than 14 days (no recovery by Day 42), the azacitidine dose may be adjusted based on bone marrow (BM) cellularity in the upcoming cycle as follows: (i) BM cellularity (15-50%): azacitidine dose adjusted to 50% of prior dose; and (ii) BM cellularity (<15%): azacitidine dose adjusted to 33% of prior dose.

It will be understood by those skilled in the art that should cytopenia occur during a 28-day cycle, for example, on day 2, 3, 4, 5, 6, 7, 8, 9, 10, 11, 12, 13, 14, 15, 16, 17, 18, 19, 20, 21, 22, 23, 24, 25, 26, or 27 of the cycle, it is not necessary to complete the full 28-day cycle prior to taking action, for example, interrupting, delaying or adjusting doses in the next cycle.

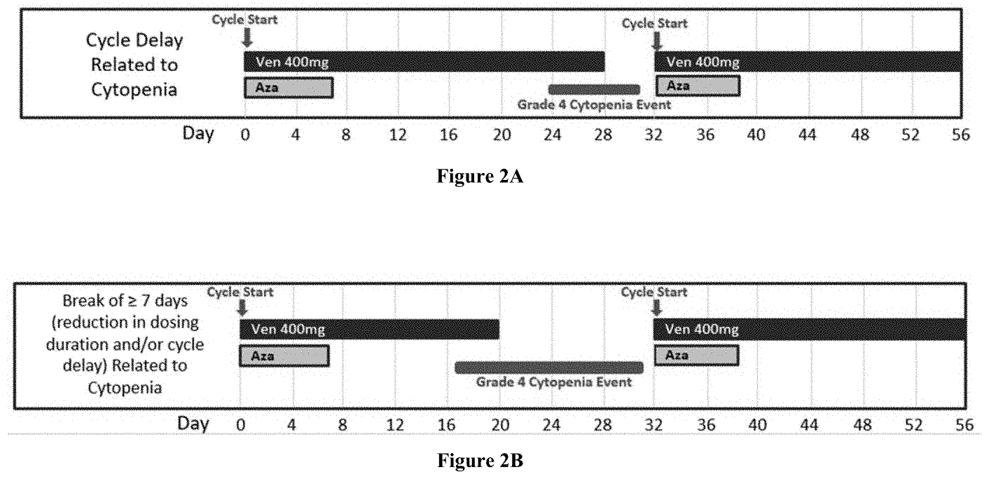

FIGS. 2A and 2B shows exemplary dose interruptions following a Grade 4 cytopenia (e.g., neutropenia and/or thrombocytopenia) event during a 28-day cycle, and resumption of the administration of venetoclax and azacitidine on the same day for the next 28-day cycle. As shown in FIG. 2A, the Grade 4 cytopenia event takes place on day 25 of a completed 28-day cycle, the patient recovers on day 31 (within 7 days), and administration of venetoclax and azacitidine is resumed on the same day for the next 28-day cycle. As shown in FIG. 2B, the Grade 4 cytopenia event takes place on day 17 of an incompleted 28-day cycle, the patient recovers on day 31 (within 14 days), and administration of venetoclax and azacitidine is resumed on the same day for the next 28-day cycle.

Also provided herein is a method of treating acute myeloid leukemia (AML) in a patient with neutropenia and/or thrombocytopenia when on a combination therapy of venetoclax and azacitidine, the method comprising:
a) administering venetoclax and azacitidine to a patient in need thereof for one or more 28-day cycles of a multiple 28-day cycle dosing regimen, wherein each 28-day cycle of the one or more 28-day cycles comprises orally administering 400 mg venetoclax on days 1-28 of the 28-day cycle, and administering azacitidine 75 mg/m² intravenously or subcutaneously on days 1-7 of the 28-day cycle; and
b) interrupting the administration of venetoclax and azacitidine to the patient after a first 28-day cycle of the one or more 28-day cycles in step (a), upon the patient having an occurrence of cytopenia, until the patient recovers from the cytopenia within 14 days from the day of the interruption, and resuming the administration of venetoclax and azacitidine for the next 28-day cycle of the one or more 28-day cycles in step (a), wherein the resumed administration of venetoclax and azacitidine starts on the same day.

In certain embodiments, the method further comprises:
c) interrupting the administration of venetoclax to the patient after a second 28-day cycle of the resumed one or more 28-day cycles in step (a), while azacitidine continues to be administered to the patient as in step (a), upon the patient having an occurrence or reoccurrence of cytopenia, until the patient recovers from the cytopenia within 14 days from the day of the interruption, and resuming the administration of venetoclax for the next 28-day cycle of the one or more 28-day cycles in step (a). In another example, the method comprises resuming the administration of venetoclax only on days 1-21 of the next 28-day cycle of the one or more 28-day cycles in step (a).

In certain embodiments, the method further comprises:
d) interrupting the administration of venetoclax to the patient after a third 28-day cycle of the resumed one or more 28-day cycles in step (a), while azacitidine continues to be administered to the patient as in step (a), upon the patient having an occurrence or reoccurrence of cytopenia, until the patient recovers from the cytopenia within 14 days from the day of the interruption, and resuming the administration of venetoclax only on days 1-21 of the next 28-day cycle of the one or more 28-day cycles in step (a);

In certain embodiments, the method further comprises:
e) reducing the dose of azacitidine intravenously or subcutaneously administered to the patient on days 1-7 of the next 28-day cycle after the fourth 28-day cycle in step (a), if the patient in step (c) or step (d) does not recover from the cytopenia within 14 days from the day of the interruption, but does recover from the cytopenia within 21 days from the day of the interruption, and resuming the 28-day cycles in step (a). In certain embodiments, the dose of azacitidine is reduced to (1) 37.5 mg/m² azacitidine when the recovered patient has a bone marrow cellularity level of between 15-50%. In certain embodiments, the dose of azacitidine is reduced to (2) 25 mg/m² azacitidine when the recovered patient has a bone marrow cellularity of less than 15%.

In certain embodiments, the patient has a bone marrow blast level of less than 5% after venetoclax and azacitidine are administered to the patient in step (a) and prior to interrupting step (b), (c) and (d).

In certain embodiments, the patient exhibits complete remission with partial hematological recovery (CRh) prior to the interrupting step (b), (c) and/or (d).

In certain embodiments, the patient exhibits complete remission with incomplete count recovery (CRi) prior to the interrupting step (b), (c) and/or (d).

In certain embodiments, the patient exhibits a morphologic leukemia-free state (MLFS) prior to the interrupting step (b), (c) and/or (d).

In certain embodiments, on days 1-3 of the first 28-day cycle of the one or more 28-day cycles step (a), the amount of venetoclax orally administered to the patient is 100 mg on day 1, 200 mg on day 2, and 400 mg on day 3.

In certain embodiments, the patient is a newly diagnosed AML patient who is ineligible for intensive chemotherapy. In certain embodiments, the patient is a newly diagnosed AML patient who is ineligible for intensive chemotherapy due to age≥75 years or ineligible for intensive chemotherapy due to comorbidities.

In certain embodiments, the cytopenia is selected from the group consisting of anemia, leukopenia, neutropenia, thrombocytopenia, and pancytopenia.

In certain embodiments, the cytopenia is neutropenia and/or thrombocytopenia.

In certain embodiments, the neutropenia is Grade 3 neutropenia or Grade 4 neutropenia, as described herein.

In certain embodiments, the thrombocytopenia is Grade 3 thrombocytopenia or Grade 4 thrombocytopenia, as described herein.

In certain embodiments, recovery from cytopenia in any of interrupting step (b), (c) and (d), is indicated when the patient has an absolute neutrophil count (“ANC”) greater than or equal to 1,500 cells/μL, 1,000 cells/μL, or 500 cells/μL. In certain embodiments, recovery from thrombocytopenia in any of interrupting step (b), (c) and (d), is indicated when the patient has a platelet count greater than or equal to 100,000 cells/μL or 50,000 cells/μL.

In certain embodiments, the administration of venetoclax and azacitidine to the patient is interrupted in step (b) after a first 28-day cycle of the one or more 28-day cycles in step (a), upon the patient having an occurrence of neutropenia, until the patient has an absolute neutrophil count (“ANC”) greater than or equal to 500 cells/μL within 14 days from the day of the interruption, upon which administration of venetoclax and azacitidine is resumed for the next 28-day cycle of the one or more 28-day cycles in step (a).

In certain embodiments, the administration of venetoclax and azacitidine to the patient is interrupted in step (b) after a first 28-day cycle of the one or more 28-day cycles in step (a), upon the patient having an occurrence of thrombocytopenia, until the patient has a platelet count greater than or equal to 50,000 cells/μL within 14 days from the day of the interruption, upon which administration of venetoclax and azacitidine is resumed for the next 28-day cycle of the one or more 28-day cycles in step (a).

In certain embodiments, the administration of venetoclax and azacitidine to the patient is interrupted in step (b) after a first 28-day cycle of the one or more 28-day cycles in step (a), upon the patient having an occurrence of neutropenia and an occurrence of thrombocytopenia, until the patient has an absolute neutrophil count (“ANC”) greater than or equal to 500 cells/μL and a platelet count greater than or equal to 50,000 cells/μL within 14 days from the day of the interruption, upon which administration of venetoclax and azacitidine is resumed for the next 28-day cycle of the one or more 28-day cycles in step (a).

In certain embodiments, the administration of venetoclax to the patient is interrupted in step (c) after a second 28-day cycle of the resumed one or more 28-day cycles in step (a), while azacitidine continues to be administered to the patient as in step (a), upon the patient having an occurrence or reoccurrence of neutropenia, until the patient has an absolute neutrophil count (“ANC”) greater than or equal to 500 cells/μL within 14 days from the day of the interruption, upon which the administration of venetoclax is resumed for the next 28-day cycle of the one or more 28-day cycles in step (a).

In certain embodiments, the administration of venetoclax to the patient is interrupted in step (c) after a second 28-day cycle of the resumed one or more 28-day cycles in step (a), while azacitidine continues to be administered to the patient as in step (a), upon the patient having an occurrence or reoccurrence of thrombocytopenia, until the patient has a platelet count greater than or equal to 50,000 cells/μL within 14 days from the day of the interruption, upon which the administration of venetoclax is resumed for the next 28-day cycle of the one or more 28-day cycles in step (a).

In certain embodiments, the administration of venetoclax to the patient is interrupted in step (c) after a second 28-day cycle of the resumed one or more 28-day cycles in step (a), while azacitidine continues to be administered to the patient as in step (a), upon the patient having an occurrence or reoccurrence of neutropenia and an occurrence or reoccurrence of thrombocytopenia, until the patient has an absolute neutrophil count (“ANC”) greater than or equal to 500 cells/μL and a platelet count greater than or equal to 50,000 cells/μL within 14 days from the day of the interruption, upon which the administration of venetoclax is resumed for the next 28-day cycle of the one or more 28-day cycles in step (a).

In certain embodiments, the administration of venetoclax to the patient is interrupted in step (d) after a third 28-day cycle of the resumed one or more 28-day cycles in step (a), while azacitidine continues to be administered to the patient as in step (a), upon the patient having an occurrence or reoccurrence of neutropenia, until the patient has an absolute neutrophil count (“ANC”) greater than or equal to 500 cells/μL within 14 days from the day of the interruption, upon which the administration of venetoclax is resumed only on days 1-21 of the next 28-day cycle of the one or more 28-day cycles in step (a).

In certain embodiments, the administration of venetoclax to the patient is interrupted in step (d) after a third 28-day cycle of the resumed one or more 28-day cycles in step (a), while azacitidine continues to be administered to the patient as in step (a), upon the patient having an occurrence or reoccurrence of thrombocytopenia, until the patient has a platelet count greater than or equal to 50,000 cells/μL within 14 days from the day of the interruption, upon which the administration of venetoclax is resumed only on days 1-21 of the next 28-day cycle of the one or more 28-day cycles in step (a).

In certain embodiments, the administration of venetoclax to the patient is interrupted in step (d) after a third 28-day cycle of the resumed one or more 28-day cycles in step (a), while azacitidine continues to be administered to the patient as in step (a), upon the patient having an occurrence or reoccurrence of neutropenia and an occurrence or reoccurrence of thrombocytopenia, until the patient has an absolute neutrophil count (“ANC”) greater than or equal to 500 cells/μL and a platelet count greater than or equal to 50,000 cells/μL within 14 days from the day of the interruption, upon which the administration of venetoclax is resumed only on days 1-21 of the next 28-day cycle of the one or more 28-day cycles in step (a).

In certain embodiments, the dose of azacitidine intravenously or subcutaneously administered to the patient in step (e) is reduced if the patient in step (c) and/or step (d) does not have an ANC greater than or equal to 500 cells/μL, within 14 days from the day of the interruption, and the patient within 21 days from the day of the interruption recovers to having an ANC greater than or equal to 500 cells/μL, and resuming the 28-day cycles in step (a). In certain embodiments, the dose of azacitidine is reduced to (1) 37.5 mg/m² azacitidine when the recovered patient has a bone marrow cellularity level of between 15-50%. In certain embodiments, the dose of azacitidine is reduced to (2) 25 mg/m² azacitidine when the recovered patient has a bone marrow cellularity of less than 15%.

In certain embodiments, the dose of azacitidine intravenously or subcutaneously administered to the patient in step (e) is reduced if the patient in step (c) and/or step (d) does not have a platelet count greater than or equal to 50,000 cells/μL, within 14 days from the day of the interruption, and the patient within 21 days from the day of the interruption recovers to having a platelet count greater than or equal to 50,000 cells/μL, and resuming the 28-day cycles in step (a). In certain embodiments, the dose of azacitidine is reduced to (1) 37.5 mg/m² azacitidine when the recovered patient has a bone marrow cellularity level of between 15-50%. In certain embodiments, the dose of azacitidine is reduced to (2) 25 mg/m² azacitidine when the recovered patient has a bone marrow cellularity of less than 15%.

In certain embodiments, the dose of azacitidine intravenously or subcutaneously administered to the patient in step (e) is reduced if the patient in step (c) and/or step (d) does not have an ANC greater than or equal to 500 cells/μL, or if the patient in step (c) and/or step (d) does not have a platelet count greater than or equal to 50,000 cells/μL, within 14 days from the day of the interruption, and the patient within 21 days from the day of the interruption recovers to having an ANC greater than or equal to 500 cells/μL and having a platelet count greater than or equal to 50,000 cells/μL, and resuming the 28-day cycles in step (a). In certain embodiments, the dose of azacitidine is reduced to (1) 37.5 mg/m² azacitidine when the recovered patient has a bone marrow cellularity level of between 15-50%. In certain embodiments, the dose of azacitidine is reduced to (2) 25 mg/m² azacitidine when the recovered patient has a bone marrow cellularity of less than 15%.

In certain embodiments, each occurrence and reoccurrence of neutropenia includes the patient having an ANC that is less than 1,000 cells/μL.

In certain embodiments, each occurrence and reoccurrence of thrombocytopenia includes the patient having a platelet count that is less than 100,000 cells/μL, less than 75,000 cells/μL, or less than 50,000 cells/μL.

In certain embodiments, each occurrence and reoccurrence of neutropenia includes the patient having a Grade 3 or Grade 4 neutropenia, and each occurrence and reoccurrence of thrombocytopenia includes the patient having a Grade 3 or Grade 4 thrombocytopenia.

In certain embodiments, the patient is in remission (e.g., has a bone marrow blast level of less than 5%) after venetoclax and azacitidine are administered to the patient in step (a) and prior to interrupting step (b), (c) and (d).

Also provided herein is a method of treating acute myeloid leukemia (AML) in a patient with neutropenia and/or thrombocytopenia when on a combination therapy of venetoclax and azacitidine, the method comprising:
a) administering venetoclax and azacitidine to a patient in need thereof for one or more 28-day cycles of a multiple 28-day cycle dosing regimen, wherein each 28-day cycle of the one or more 28-day cycles comprises orally administering 400 mg venetoclax on days 1-28 of the 28-day cycle, and administering azacitidine 75 mg/m² intravenously or subcutaneously on days 1-7 of the 28-day cycle; and
b) interrupting the administration of venetoclax and azacitidine to the patient after a first 28-day cycle of the one or more 28-day cycles in step (a), upon the patient having:
   (i) an occurrence of neutropenia, until the patient has an ANC greater than or equal to 500 cells/μL within 14 days from the day of the interruption, and/or
   (ii) an occurrence of thrombocytopenia, until the patient has a platelet count greater than or equal to 50,000 cells/μL within 14 days from the day of the interruption; and resuming the administration of venetoclax and azacitidine for the next 28-day cycle of the one or more 28-day cycles in step (a), wherein the resumed administration of venetoclax and azacitidine starts on the same day.

In certain embodiments, the method further comprises:
c) interrupting the administration of venetoclax to the patient after a second 28-day cycle of the resumed one or more 28-day cycles in step (a), while azacitidine continues to be administered to the patient as in step (a), upon the patient having:
   (i) an occurrence or reoccurrence of neutropenia, until the patient has an ANC greater than or equal to 500 cells/μL within 14 days from the day of the interruption, and/or
   (ii) an occurrence or reoccurrence of thrombocytopenia, until the patient has a platelet count greater than or equal to 50,000 cells/μL within 14 days from the day of the interruption; and resuming the administration of venetoclax for the next 28-day cycle of the one or more 28-day cycles in step (a).

In certain embodiments, the method further comprises:
d) interrupting the administration of venetoclax to the patient after a third 28-day cycle of the resumed one or more 28-day cycles in step (a), while azacitidine continues to be administered to the patient as in step (a), upon the patient having:
   (i) an occurrence or reoccurrence of neutropenia, until the patient has an ANC greater than or equal to 500 cells/μL within 14 days from the day of the interruption, and/or
   (ii) an occurrence or reoccurrence of thrombocytopenia, until the patient has a platelet count greater than or equal to 50,000 cells/μL within 14 days from the day of the interruption; and resuming the administration of venetoclax only on days 1-21 of the next 28-day cycle of the one or more 28-day cycles in step (a);

In certain embodiments, the method further comprises:
e) reducing the dose of azacitidine intravenously or subcutaneously administered to the patient on days 1-7 of the next 28-day cycle after the fourth 28-day cycle in step (a), if the patient in step (c) and/or step (d) does not have an ANC greater than or equal to 500 cells/4, or if the patient in step (c) and/or step (d) does not have a platelet count greater than or equal to 50,000 cells/μL, within 14 days from the day of the interruption, and the patient within 21 days from the day of the interruption recovers to having an ANC greater than or equal to 500 cells/μL and having a platelet count greater than or equal to 50,000 cells/μL, and resuming the 28-day cycles in step (a) wherein the administered dose of azacitidine is reduced to (1) 37.5 mg/m² azacitidine when the recovered patient has a bone marrow cellularity level of between 15-50%, or (2) 25 mg/m² azacitidine when the recovered patient has a bone marrow cellularity of less than 15%.

In certain embodiments, the occurrence or reoccurrence of neutropenia in step (c) and/or step (d) lasts for at least one week and is not due to a relapse of AML.

In certain embodiments, each occurrence and reoccurrence of neutropenia includes the patient having an ANC that is less than 1,000 cells/μL.

In certain embodiments, each occurrence and reoccurrence of thrombocytopenia includes the patient having a platelet count that is less than 100,000 cells/μL, less than 75,000 cells/μL, or less than 50,000 cells/μL.

In certain embodiments, each occurrence and reoccurrence of neutropenia includes the patient having a Grade 3 or Grade 4 neutropenia, and each occurrence and reoccurrence of thrombocytopenia includes the patient having a Grade 3 or Grade 4 thrombocytopenia.

In certain embodiments, the patient has a bone marrow blast level of less than 5% after venetoclax and azacitidine are administered to the patient in step (a) and prior to interrupting step (b), (c) and (d).

In certain embodiments, on days 1-3 of the first 28-day cycle of the one or more 28-day cycles step (a), the amount of venetoclax orally administered to the patient is 100 mg on day 1, 200 mg on day 2, and 400 mg on day 3.

In certain embodiments, the patient is a newly diagnosed AML patient who is ineligible for intensive chemotherapy. In certain embodiments, the patient is a newly diagnosed AML patient who is ineligible for intensive chemotherapy due to age≥75 years or ineligible for intensive chemotherapy due to comorbidities.

Also provided herein is a method of treating acute myeloid leukemia (AML) in a patient with neutropenia and/or thrombocytopenia when on a combination therapy of venetoclax and azacitidine, the method comprising:
a) administering venetoclax and azacitidine to a patient in need thereof for one or more 28-day cycles of a multiple 28-day cycle dosing regimen, wherein each 28-day cycle of the one or more 28-day cycles comprises orally administering 400 mg venetoclax on days 1-28 of the 28-day cycle, and administering azacitidine 75 mg/m² intravenously or subcutaneously on days 1-7 of the 28-day cycle; and
b) interrupting the administration of venetoclax and azacitidine to the patient after a first 28-day cycle of the one or more 28-day cycles in step (a), upon the patient having an occurrence of neutropenia, until the patient has an absolute neutrophil count (“ANC”) greater than or equal to 500 cells/μL within 14 days from the day of the interruption, and
   resuming the administration of venetoclax and azacitidine for the next 28-day cycle of the one or more 28-day cycles in step (a), wherein the resumed administration of venetoclax and azacitidine starts on the same day.

In certain embodiments, the method further comprises:
c) interrupting the administration of venetoclax to the patient after a second 28-day cycle of the resumed one or more 28-day cycles in step (a), while azacitidine continues to be administered to the patient as in step (a), upon the patient having a reoccurrence of neutropenia, until the patient has an ANC greater than or equal to 500 cells/μL within 14 days from the day of the interruption, and
resuming the administration of venetoclax for the next 28-day cycle of the one or more 28-day cycles in step (a).

In certain embodiments, the method further comprises:
d) interrupting the administration of venetoclax to the patient after a third 28-day cycle of the resumed one or more 28-day cycles in step (a), while azacitidine continues to be administered to the patient as in step (a), upon the patient having:
   (i) a reoccurrence of neutropenia, until the patient has an ANC greater than or equal to 500 cells/μL within 14 days from the day of the interruption, and/or
   (ii) an occurrence of thrombocytopenia, until the patient has a platelet count greater than or equal to 50,000 cells/μL within 14 days from the day of the interruption; and
resuming the administration of venetoclax only on days 1-21 of the next 28-day cycle of the one or more 28-day cycles in step (a);

In certain embodiments, the method further comprises:
e) reducing the dose of azacitidine intravenously or subcutaneously administered to the patient on days 1-7 of the next 28-day cycle after the fourth 28-day cycle in step (a), if the patient in step (c) or step (d) does not have an ANC greater than or equal to 500 cells/μL, or if the patient in step (d) does not have a platelet count greater than or equal to 50,000 cells/μL, within 14 days from the day of the interruption, and the patient within 21 days from the day of the interruption recovers to having an ANC greater than or equal to 500 cells/μL and having a platelet count greater than or equal to 50,000 cells/μL, and
resuming the 28-day cycles in step (a) wherein the administered dose of azacitidine is reduced to (1) 37.5 mg/m² azacitidine when the recovered patient has a bone marrow cellularity level of between 15-50%, or (2) 25 mg/m² azacitidine when the recovered patient has a bone marrow cellularity of less than 15%.

In certain embodiments, the occurrence or reoccurrence of neutropenia in step (c) and/or step (d) lasts for at least one week and is not due to a relapse of AML.

In certain embodiments, each occurrence and reoccurrence of neutropenia includes the patient having an ANC that is less than 1,000 cells/μL.

In certain embodiments, each occurrence and reoccurrence of thrombocytopenia includes the patient having a platelet count that is less than 100,000 cells/μL, less than 75,000 cells/μL, or less than 50,000 cells/μL.

In certain embodiments, each occurrence and reoccurrence of neutropenia includes the patient having a Grade 3 or Grade 4 neutropenia, and each occurrence and reoccurrence of thrombocytopenia includes the patient having a Grade 3 or Grade 4 thrombocytopenia.

In certain embodiments, the patient has a bone marrow blast level of less than 5% after venetoclax and azacitidine are administered to the patient in step (a) and prior to interrupting step (b), (c) and (d).

In certain embodiments, on days 1-3 of the first 28-day cycle of the one or more 28-day cycles step (a), the amount of venetoclax orally administered to the patient is 100 mg on day 1, 200 mg on day 2, and 400 mg on day 3.

In certain embodiments, the patient is a newly diagnosed AML patient who is ineligible for intensive chemotherapy. In certain embodiments, the patient is a newly diagnosed AML patient who is ineligible for intensive chemotherapy due to age≥75 years or ineligible for intensive chemotherapy due to comorbidities.

Also provided herein is a method of treating acute myeloid leukemia (AML) in a patient with neutropenia and/or thrombocytopenia when on a combination therapy of venetoclax and azacitidine, the method comprising:
a) administering venetoclax and azacitidine to a patient in need thereof for one or more 28-day cycles of a multiple 28-day cycle dosing regimen, wherein each 28-day cycle of the one or more 28-day cycles comprises orally administering 400 mg venetoclax on days 1-28 of the 28-day cycle, and administering azacitidine 75 mg/m² intravenously or subcutaneously on days 1-7 of the 28-day cycle;
b) interrupting the administration of venetoclax and azacitidine to the patient after a first 28-day cycle of the one or more 28-day cycles in step (a), upon the patient having:
   (i) an occurrence of neutropenia, until the patient has absolute neutrophil count (“ANC”) greater than or equal to 500 cells/μL within 14 days from the day of the interruption, and/or
   (ii) an occurrence of thrombocytopenia, until the patient has a platelet count greater than or equal to 50,000 cells/μL within 14 days from the day of the interruption; and
resuming the administration of venetoclax and azacitidine for the next 28-day cycle of the one or more 28-day cycles in step (a), wherein the resumed administration of venetoclax and azacitidine starts on the same day;
c) interrupting the administration of venetoclax to the patient after a second 28-day cycle of the resumed one or more 28-day cycles in step (a), while azacitidine continues to be administered to the patient as in step (a), upon the patient having:
   (i) an occurrence or reoccurrence of neutropenia, until the patient has absolute neutrophil count (“ANC”) greater than or equal to 500 cells/μL within 14 days from the day of the interruption, and/or
   (ii) an occurrence or reoccurrence of thrombocytopenia, until the patient has a platelet count greater than or equal to 50,000 cells/μL within 14 days from the day of the interruption; and resuming the administration of venetoclax for the next 28-day cycle of the one or more 28-day cycles in step (a);
d) interrupting the administration of venetoclax to the patient after a third 28-day cycle of the resumed one or more 28-day cycles in step (a), while azacitidine continues to be administered to the patient as in step (a), upon the patient having:
   (i) a reoccurrence or reoccurrence of neutropenia, until the patient has an ANC greater than or equal to 500 cells/μL within 14 days from the day of the interruption, and/or
   (ii) an occurrence or reoccurrence of thrombocytopenia, until the patient has a platelet count greater than or equal to 50,000 cells/μL within 14 days from the day of the interruption; and resuming the administration of venetoclax only on days 1-21 of the next 28-day cycle of the one or more 28-day cycles in step (a); and
e) reducing the dose of azacitidine intravenously or subcutaneously administered to the patient on days 1-7 of the next 28-day cycle after the fourth 28-day cycle in step (a), if the patient in step (c) and/or step (d) does not have an ANC greater than or equal to 500 cells/μL, or if the patient in step (c) and/or step (d) does not have a platelet count greater than or equal to 50,000 cells/μL, within 14 days from the day of the interruption, and the patient within 21 days from the day of the interruption recovers to having an ANC greater than or equal to 500 cells/μL and having a platelet count greater than or equal to 50,000 cells/μL, and resuming the 28-day cycles in step (a) wherein the administered dose of azacitidine is reduced to (1) 37.5 mg/m² azacitidine when the recovered patient has a bone marrow cellularity level of between 15-50%, or (2) 25 mg/m² azacitidine when the recovered patient has a bone marrow cellularity of less than 15%.

In certain embodiments, the occurrence or reoccurrence of neutropenia in step (c) and/or step (d) lasts for at least one week and is not due to a relapse of AML.

In certain embodiments, each occurrence and reoccurrence of neutropenia includes the patient having an ANC that is less than 1,000 cells/μL.

In certain embodiments, each occurrence and reoccurrence of thrombocytopenia includes the patient having a platelet count that is less than 100,000 cells/μL, less than 75,000 cells/μL, or less than 50,000 cells/μL.

In certain embodiments, each occurrence and reoccurrence of neutropenia includes the patient having a Grade 3 or Grade 4 neutropenia, and each occurrence and reoccurrence of thrombocytopenia includes the patient having a Grade 3 or Grade 4 thrombocytopenia.

In certain embodiments, the patient has a bone marrow blast level of less than 5% after venetoclax and azacitidine are administered to the patient in step (a) and prior to interrupting step (b), (c) and (d).

In certain embodiments, on days 1-3 of the first 28-day cycle of the one or more 28-day cycles step (a), the amount of venetoclax orally administered to the patient is 100 mg on day 1, 200 mg on day 2, and 400 mg on day 3.

In certain embodiments, the patient is a newly diagnosed AML, patient who is ineligible for intensive chemotherapy. In certain embodiments, the patient is a newly diagnosed AML patient who is ineligible for intensive chemotherapy due to age≥75 years or ineligible for intensive chemotherapy due to comorbidities.

Also provided herein is a method of treating acute myeloid leukemia (AML) in a patient with neutropenia and/or thrombocytopenia when on a combination therapy of venetoclax and azacitidine, the method comprising:
a) administering venetoclax and azacitidine to a patient in need thereof for one or more 28-day cycles of a multiple 28-day cycle dosing regimen, wherein each 28-day cycle of the one or more 28-day cycles comprises orally administering 400 mg venetoclax on days 1-28 of the 28-day cycle, and administering azacitidine 75 mg/m² intravenously or subcutaneously on days 1-7 of the 28-day cycle;
b) interrupting the administration of venetoclax and azacitidine to the patient after a first 28-day cycle of the one or more 28-day cycles in step (a), upon the patient having an occurrence of Grade 4 neutropenia, until the patient has an absolute neutrophil count (“ANC”) greater than or equal to 500 cells/μL within 14 days from the day of the interruption, and
   resuming the administration of venetoclax and azacitidine for the next 28-day cycle of the one or more 28-day cycles in step (a), wherein the resumed administration of venetoclax and azacitidine starts on the same day;
c) interrupting the administration of venetoclax to the patient after a second 28-day cycle of the resumed one or more 28-day cycles in step (a), while azacitidine continues to be administered to the patient as in step (a), upon the patient having a reoccurrence of Grade 4 neutropenia, until the patient has an ANC greater than or equal to 500 cells/μL within 14 days from the day of the interruption, and resuming the administration of venetoclax for the next 28-day cycle of the one or more 28-day cycles in step (a);
d) interrupting the administration of venetoclax to the patient after a third 28-day cycle of the resumed one or more 28-day cycles in step (a), while azacitidine continues to be administered to the patient as in step (a), upon the patient having:
   (i) a reoccurrence of Grade 4 neutropenia, until the patient has an ANC greater than or equal to 500 cells/μL within 14 days from the day of the interruption, and/or
   (ii) an occurrence of thrombocytopenia, until the patient has a platelet count greater than or equal to 50,000 cells/μL within 14 days from the day of the interruption; and resuming the administration of venetoclax only on days 1-21 of the next 28-day cycle of the one or more 28-day cycles in step (a);
e) reducing the dose of azacitidine intravenously or subcutaneously administered to the patient on days 1-7 of the next 28-day cycle after the fourth 28-day cycle in step (a), if the patient in step (c) or step (d) does not have an ANC greater than or equal to 500 cells/μL, or if the patient in step (d) does not have a platelet count greater than or equal to 50,000 cells/μL, within 14 days from the day of the interruption, and the patient within 21 days from the day of the interruption recovers to having an ANC greater than or equal to 500 cells/μL and having a platelet count greater than or equal to 50,000 cells/μL, and
resuming the 28-day cycles in step (a) wherein the administered dose of azacitidine is reduced to (1) 37.5 mg/m² azacitidine when the recovered patient has a bone marrow cellularity level of between 15-50%, or (2) 25 mg/m² azacitidine when the recovered patient has a bone marrow cellularity of less than 15%.

In certain embodiments, the occurrence or reoccurrence of neutropenia in step (c) and/or step (d) lasts for at least one week and is not due to a relapse of AML.

In certain embodiments, each occurrence and reoccurrence of neutropenia includes the patient having an ANC that is less than 1,000 cells/μL.

In certain embodiments, each occurrence and reoccurrence of thrombocytopenia includes the patient having a platelet count that is less than 100,000 cells/μL, less than 75,000 cells/μL, or less than 50,000 cells/μL.

In certain embodiments, each occurrence and reoccurrence of neutropenia includes the patient having a Grade 3 or Grade 4 neutropenia, and each occurrence and reoccurrence of thrombocytopenia includes the patient having a Grade 3 or Grade 4 thrombocytopenia.

In certain embodiments, the patient has a bone marrow blast level of less than 5% after venetoclax and azacitidine are administered to the patient in step (a) and prior to interrupting step (b), (c) and (d).

In certain embodiments, on days 1-3 of the first 28-day cycle of the one or more 28-day cycles step (a), the amount of venetoclax orally administered to the patient is 100 mg on day 1, 200 mg on day 2, and 400 mg on day 3.

In certain embodiments, the patient is a newly diagnosed AML, patient who is ineligible for intensive chemotherapy. In certain embodiments, the patient is a newly diagnosed AML patient who is ineligible for intensive chemotherapy due to age≥75 years or ineligible for intensive chemotherapy due to comorbidities.

In another aspect, provided herein is a method of managing cytopenia in a patient with neutropenia and/or thrombocytopenia on a combination therapy of venetoclax and azacitidine to treat acute myeloid leukemia (AML), the method comprising: interrupting administration of venetoclax and azacitidine to a patient in need thereof after a first cycle of one or more administration cycles, upon the patient having an occurrence of cytopenia, until the patient recovers from the cytopenia, and resuming the administration of venetoclax and azacitidine for the next cycle of the one or more cycles, wherein the resumed administration of venetoclax and azacitidine starts on the same day.

In certain embodiments, the cytopenia is selected from the group consisting of anemia, leukopenia, neutropenia, thrombocytopenia, and pancytopenia.

In certain embodiments, the cytopenia is neutropenia and/or thrombocytopenia.

In certain embodiments, the neutropenia is Grade 3 neutropenia or Grade 4 neutropenia, as described herein.

In certain embodiments, the thrombocytopenia is Grade 1 thrombocytopenia or Grade 2 thrombocytopenia, as described herein.

In certain embodiments, recovery from cytopenia in any of interrupting step (b), (c) and (d), is indicated when the patient has an absolute neutrophil count (“ANC”) greater than or equal to 1,500 cells/4, 1,000 cells/4, or 500 cells/μL. In certain embodiments, recovery from thrombocytopenia in any of interrupting step (b), (c) and (d), is indicated when the patient has a platelet count greater than or equal to 100,000 cells/μL or 50,000 cells/μL.

In certain embodiments, the method of managing cytopenia comprises:
a) administering venetoclax and azacitidine to a patient in need thereof for one or more 28-day cycles of a multiple 28-day cycle dosing regimen, wherein each 28-day cycle of the one or more 28-day cycles comprises orally administering 400 mg venetoclax on days 1-28 of the 28-day cycle, and administering azacitidine 75 mg/m² intravenously or subcutaneously on days 1-7 of the 28-day cycle; and
b) interrupting the administration of venetoclax and azacitidine to the patient after a first 28-day cycle of the one or more 28-day cycles in step (a), upon the patient having:
   (i) an occurrence of neutropenia, until the patient has absolute neutrophil count (“ANC”) greater than or equal to 500 cells/μL within 14 days from the day of the interruption, and/or
   (ii) an occurrence of thrombocytopenia, until the patient has a platelet count greater than or equal to 50,000 cells/μL within 14 days from the day of the interruption;
resuming the administration of venetoclax and azacitidine for the next 28-day cycle of the one or more 28-day cycles in step (a), wherein the resumed administration of venetoclax and azacitidine starts on the same day.

In certain embodiments, the method further comprises:
c) interrupting the administration of venetoclax to the patient after a second 28-day cycle of the resumed one or more 28-day cycles in step (a), while azacitidine continues to be administered to the patient as in step (a), upon the patient having:
   (i) an occurrence or reoccurrence of neutropenia, until the patient has absolute neutrophil count (“ANC”) greater than or equal to 500 cells/μL within 14 days from the day of the interruption, and/or
   (ii) an occurrence or reoccurrence of thrombocytopenia, until the patient has a platelet count greater than or equal to 50,000 cells/μL within 14 days from the day of the interruption; and
resuming the administration of venetoclax for the next 28-day cycle of the one or more 28-day cycles in step (a).

In certain embodiments, the method further comprises:
d) interrupting the administration of venetoclax to the patient after a third 28-day cycle of the resumed one or more 28-day cycles in step (a), while azacitidine continues to be administered to the patient as in step (a), upon the patient having:
   (i) an occurrence or reoccurrence of neutropenia, until the patient has an ANC greater than or equal to 500 cells/μL within 14 days from the day of the interruption, and/or
   (ii) an occurrence or reoccurrence of thrombocytopenia, until the patient has a platelet count greater than or equal to 50,000 cells/μL within 14 days from the day of the interruption; and
resuming the administration of venetoclax only on days 1-21 of the next 28-day cycle of the one or more 28-day cycles in step (a);

In certain embodiments, the method further comprises:
e) reducing the dose of azacitidine intravenously or subcutaneously administered to the patient on days 1-7 of the next 28-day cycle after the fourth 28-day cycle in step (a), if the patient in step (c) and/or step (d) does not have an ANC greater than or equal to 500 cells/μL, or if the patient in step (c) and/or step (d) does not have a platelet count greater than or equal to 50,000 cells/μL, within 14 days from the day of the interruption, and the patient within 21 days from the day of the interruption recovers to having an ANC greater than or equal to 500 cells/μL and having a platelet count greater than or equal to 50,000 cells/μL, and
resuming the 28-day cycles in step (a) wherein the administered dose of azacitidine is reduced to (1) 37.5 mg/m² azacitidine when the recovered patient has a bone marrow cellularity level of between 15-50%, or (2) 25 mg/m² azacitidine when the recovered patient has a bone marrow cellularity of less than 15%.

In certain embodiments, the occurrence or reoccurrence of neutropenia in step (c) and/or step (d) lasts for at least one week and is not due to a relapse of AML.

In certain embodiments, each occurrence and reoccurrence of neutropenia includes the patient having an ANC that is less than 1,000 cells/μL.

In certain embodiments, each occurrence and reoccurrence of thrombocytopenia includes the patient having a platelet count that is less than 100,000 cells/μL, less than 75,000 cells/μL, or less than 50,000 cells/μL.

In certain embodiments, each occurrence and reoccurrence of neutropenia includes the patient having a Grade 3 or Grade 4 neutropenia, and each occurrence and reoccurrence of thrombocytopenia includes the patient having a Grade 3 or Grade 4 thrombocytopenia.

In certain embodiments, the patient has a bone marrow blast level of less than 5% after venetoclax and azacitidine are administered to the patient in step (a) and prior to interrupting step (b), (c) and (d).

In certain embodiments, on days 1-3 of the first 28-day cycle of the one or more 28-day cycles step (a), the amount of venetoclax orally administered to the patient is 100 mg on day 1, 200 mg on day 2, and 400 mg on day 3.

In certain embodiments, the patient is a newly diagnosed AML, patient who is ineligible for intensive chemotherapy. In certain embodiments, the patient is a newly diagnosed AML patient who is ineligible for intensive chemotherapy due to age≥75 years or ineligible for intensive chemotherapy due to comorbidities.

Also provided herein is a method of managing cytopenia in a patient with neutropenia and/or thrombocytopenia on a combination therapy of venetoclax and azacitidine to treat acute myeloid leukemia (AML), the method comprising:
a) administering venetoclax and azacitidine to a patient in need thereof for one or more 28-day cycles of a multiple 28-day cycle dosing regimen, wherein each 28-day cycle of the one or more 28-day cycles comprises orally administering 400 mg venetoclax on days 1-28 of the 28-day cycle, and administering azacitidine 75 mg/m² intravenously or subcutaneously on days 1-7 of the 28-day cycle;
b) interrupting the administration of venetoclax and azacitidine to the patient after a first 28-day cycle of the one or more 28-day cycles in step (a), upon the patient having:
   (i) an occurrence of neutropenia, until the patient has absolute neutrophil count (“ANC”) greater than or equal to 500 cells/μL within 14 days from the day of the interruption, and/or
   (ii) an occurrence of thrombocytopenia, until the patient has a platelet count greater than or equal to 50,000 cells/μL within 14 days from the day of the interruption; and resuming the administration of venetoclax and azacitidine for the next 28-day cycle of the one or more 28-day cycles in step (a), wherein the resumed administration of venetoclax and azacitidine starts on the same day;
c) interrupting the administration of venetoclax to the patient after a second 28-day cycle of the resumed one or more 28-day cycles in step (a), while azacitidine continues to be administered to the patient as in step (a), upon the patient having:
   (i) an occurrence or reoccurrence of neutropenia, until the patient has absolute neutrophil count (“ANC”) greater than or equal to 500 cells/μl, within 14 days from the day of the interruption, and/or
   (ii) an occurrence or reoccurrence of thrombocytopenia, until the patient has a platelet count greater than or equal to 50,000 cells/μl, within 14 days from the day of the interruption; and
   resuming the administration of venetoclax for the next 28-day cycle of the one or more 28-day cycles in step (a);
d) interrupting the administration of venetoclax to the patient after a third 28-day cycle of the resumed one or more 28-day cycles in step (a), while azacitidine continues to be administered to the patient as in step (a), upon the patient having:
   (i) an occurrence or reoccurrence of neutropenia, until the patient has an ANC greater than or equal to 500 cells/μl, within 14 days from the day of the interruption, and/or
   (ii) an occurrence or reoccurrence of thrombocytopenia, until the patient has a platelet count greater than or equal to 50,000 cells/μl, within 14 days from the day of the interruption; and
   resuming the administration of venetoclax only on days 1-21 of the next 28-day cycle of the one or more 28-day cycles in step (a); and
e) reducing the dose of azacitidine intravenously or subcutaneously administered to the patient on days 1-7 of the next 28-day cycle after the fourth 28-day cycle in step (a), if the patient in step (c) and/or step (d) does not have an ANC greater than or equal to 500 cells/μL, or if the patient in step (c) and/or step (d) does not have a platelet count greater than or equal to 50,000 cells/μL, within 14 days from the day of the interruption, and the patient within 21 days from the day of the interruption recovers to having an ANC greater than or equal to 500 cells/μl, and having a platelet count greater than or equal to 50,000 cells/μL, and
   resuming the 28-day cycles in step (a) wherein the administered dose of azacitidine is reduced to (1) 37.5 mg/m² azacitidine when the recovered patient has a bone marrow cellularity level of between 15-50%, or (2) 25 mg/m² azacitidine when the recovered patient has a bone marrow cellularity of less than 15%.

In certain embodiments, the occurrence or reoccurrence of neutropenia in step (c) and/or step (d) lasts for at least one week and is not due to a relapse of AML.

In certain embodiments, each occurrence and reoccurrence of neutropenia includes the patient having an ANC that is less than 1,000 cells/μL.

In certain embodiments, each occurrence and reoccurrence of thrombocytopenia includes the patient having a platelet count that is less than 100,000 cells/μL, less than 75,000 cells/μL, or less than 50,000 cells/μL.

In certain embodiments, each occurrence and reoccurrence of neutropenia includes the patient having a Grade 3 or Grade 4 neutropenia, and each occurrence and reoccurrence of thrombocytopenia includes the patient having a Grade 3 or Grade 4 thrombocytopenia.

In certain embodiments, the patient has a bone marrow blast level of less than 5% after venetoclax and azacitidine are administered to the patient in step (a) and prior to interrupting step (b), (c) and (d). In certain embodiments, on days 1-3 of the first 28-day cycle of the one or more 28-day cycles step (a), the amount of venetoclax orally administered to the patient is 100 mg on day 1, 200 mg on day 2, and 400 mg on day 3.

In certain embodiments, the patient is a newly diagnosed AML patient who is ineligible for intensive chemotherapy. In certain embodiments, the patient is a newly diagnosed AML patient who is ineligible for intensive chemotherapy due to age≥75 years or ineligible for intensive chemotherapy due to comorbidities.

Also provided herein is a method of managing cytopenia in a patient with neutropenia and/or thrombocytopenia on a combination therapy of venetoclax and azacitidine to treat acute myeloid leukemia (AML), the method comprising:
a) administering venetoclax and azacitidine to a patient in need thereof for one or more 28-day cycles of a multiple 28-day cycle dosing regimen, wherein each 28-day cycle of the one or more 28-day cycles comprises orally administering 400 mg venetoclax on days 1-28 of the 28-day cycle, and administering azacitidine 75 mg/m² intravenously or subcutaneously on days 1-7 of the 28-day cycle;
b) interrupting the administration of venetoclax and azacitidine to the patient after a first 28-day cycle of the one or more 28-day cycles in step (a), upon the patient having an occurrence of Grade 4 neutropenia, until the patient has an absolute neutrophil count (“ANC”) greater than or equal to 500 cells/μl, within 14 days from the day of the interruption, and
   resuming the administration of venetoclax and azacitidine for the next 28-day cycle of the one or more 28-day cycles in step (a), wherein the resumed administration of venetoclax and azacitidine starts on the same day;
c) interrupting the administration of venetoclax to the patient after a second 28-day cycle of the resumed one or more 28-day cycles in step (a), while azacitidine continues to be administered to the patient as in step (a), upon the patient having a reoccurrence of Grade 4 neutropenia, until the patient has an ANC greater than or equal to 500 cells/μl, within 14 days from the day of the interruption, and resuming the administration of venetoclax for the next 28-day cycle of the one or more 28-day cycles in step (a);
d) interrupting the administration of venetoclax to the patient after a third 28-day cycle of the resumed one or more 28-day cycles in step (a), while azacitidine continues to be administered to the patient as in step (a), upon the patient having:
   (i) a reoccurrence of Grade 4 neutropenia, until the patient has an ANC greater than or equal to 500 cells/μl, within 14 days from the day of the interruption, and/or
   (ii) an occurrence of thrombocytopenia, until the patient has a platelet count greater than or equal to 50,000 cells/μl, within 14 days from the day of the interruption; and
   resuming the administration of venetoclax only on days 1-21 of the next 28-day cycle of the one or more 28-day cycles in step (a);
e) reducing the dose of azacitidine intravenously or subcutaneously administered to the patient on days 1-7 of the next 28-day cycle after the fourth 28-day cycle in step (a), if the patient in step (c) or step (d) does not have an ANC greater than or equal to 500 cells/μL, or if the patient in step (d) does not have a platelet count greater than or equal to 50,000 cells/μL, within 14 days from the day of the interruption, and the patient within 21 days from the day of the interruption recovers to having an ANC greater than or equal to 500 cells/μL and having a platelet count greater than or equal to 50,000 cells/μL, and
resuming the 28-day cycles in step (a) wherein the administered dose of azacitidine is reduced to (1) 37.5 mg/m² azacitidine when the recovered patient has a bone marrow cellularity level of between 15-50%, or (2) 25 mg/m² azacitidine when the recovered patient has a bone marrow cellularity of less than 15%.

In certain embodiments, the occurrence or reoccurrence of neutropenia in step (c) and/or step (d) lasts for at least one week and is not due to a relapse of AML.

In certain embodiments, each occurrence and reoccurrence of neutropenia includes the patient having an ANC that is less than 1,000 cells/μL.

In certain embodiments, each occurrence and reoccurrence of thrombocytopenia includes the patient having a platelet count that is less than 100,000 cells/μL, less than 75,000 cells/μL, or less than 50,000 cells/μL.

In certain embodiments, each occurrence and reoccurrence of neutropenia includes the patient having a Grade 3 or Grade 4 neutropenia, and each occurrence and reoccurrence of thrombocytopenia includes the patient having a Grade 3 or Grade 4 thrombocytopenia.

In certain embodiments, the patient has a bone marrow blast level of less than 5% after venetoclax and azacitidine are administered to the patient in step (a) and prior to interrupting step (b), (c) and (d).

In certain embodiments, on days 1-3 of the first 28-day cycle of the one or more 28-day cycles step (a), the amount of venetoclax orally administered to the patient is 100 mg on day 1, 200 mg on day 2, and 400 mg on day 3.

In certain embodiments, the patient is a newly diagnosed AML patient who is ineligible for intensive chemotherapy. In certain embodiments, the patient is a newly diagnosed AML patient who is ineligible for intensive chemotherapy due to age≥75 years or ineligible for intensive chemotherapy due to comorbidities.

In certain embodiments, the method disclosed herein further comprises administering Granulocyte colony-stimulating factor (G-CSF) to the patient as part of managing the post-remission cytopenia.

In certain embodiments, the methods provided herein include a treating cycle delay, a treatment interruption, or a dosage reduction, or combinations thereof.

In certain embodiments, the methods provided herein manages cytopenia in the patient without negatively impacting a treatment outcome, such as overall survival (OS) efficacy outcome, in said patient.

In certain embodiments, the methods provided herein manages cytopenia in the patient and positively impacts a treatment outcome, such as overall survival (OS) efficacy outcome, in said patient. For example, in certain embodiments, the methods provided herein manages cytopenia in the patient and improves a treatment outcome, such as improves or extends overall survival (OS) efficacy outcome, in said patient.

Also provided herein are formulations of venetoclax and azacitidine for use in the methods described herein.

For example, venetoclax may be administered in a formulation according to standard practices known by those skilled in the art, e.g., as described in the prescribing information for venetoclax. For example, venetoclax may be administered in tablet form containing 100 mg venetoclax as the active ingredient. Four tablets may be taken to obtain a 400 mg venetoclax dose. See Prescribing Information for VENCLEXTA® (venetoclax) tablets, for oral use, revised November 2020, herein incorporated by reference in its entirety.

For example, azacitidine may be administered in a formulation according to standard practices known by those skilled in the art, e.g., as described in the prescribing information for azacitidine. For example, 75 mg/m² azacitidine may be administered by subcutaneous or intravenous injection. See Prescribing Information for VIDAZA® (azacitidine for injection) for subcutaneous or intravenous use, revised March 2020, herein incorporated by reference in its entirety.

The following examples are provided to aid the understanding of the present disclosure, the true scope of which is set forth in the appended claims. It is understood that modifications can be made in the procedures set forth without departing from the spirit of the disclosure.

### 7. EXAMPLES

### 7.1 Example 1: A Randomized, Double-Blind, Placebo Controlled Phase 3 Study of Venetoclax in Combination with Azacitidine Versus Azacitidine in Treatment Naïve Subjects with Acute Myeloid Leukemia Who are Ineligible for Standard Induction Therapy

Study Population: Adult male and female subjects, with confirmed AML, who are treatment naïve for AML, with a projected life expectancy of at least 12 weeks and be ineligible for treatment with a standard cytarabine and anthracycline induction regimen due to age or co-morbidities.

Methodology: This randomized, double-blind, placebo controlled trial evaluates the efficacy of venetoclax in combination with azacitidine versus placebo in combination with azacitidine in treating naïve subjects with AML who are ≥18 years of age and not eligible for standard induction therapy due to age or co-morbidities. If a subject enrolled on any arm achieves complete remission with incomplete marrow recovery (CRi) or has morphologic leukemia free bone marrow after completion of Cycle 1, venetoclax/placebo may be interrupted from Day 29 for up to 14 days or until recovery of ANC≥500/μL. If venetoclax/placebo is interrupted, then Cycle 2 administration of azacitidine will also be delayed. Cycle 2 treatment with venetoclax/placebo and azacitidine will resume on the same day after the interruption. Subjects with resistant disease after the end of Cycle 1 will need a bone marrow aspirate to evaluate response after completion of Cycle 2 or 3 if hematologic improvement is seen. A bone marrow aspirate and biopsy will be done at the end of Cycle 4 and every 3 cycles after for subjects with resistant disease until two successive samples indicate CR or CRi. For subjects with a response of CRi a repeat bone marrow aspirate must be performed to confirm a CR once peripheral blood count recovery is noted. For subjects who have not recovered ANC≥500 μL within 14 days of drug interruption or require longer duration of interruption between treatment cycles, bone marrow aspirate may be performed per investigator discretion to assess disease status before resuming treatment with next Cycle. Blood and bone marrow samples will be collected for biomarker analysis and exploratory research at designated timepoints throughout the study.

Screening: Unless otherwise specified, screening procedures must be performed within 21 days prior to randomization. Once screening procedures are complete and eligibility is confirmed, subjects will be randomized using a 2:1 ratio to one of the following two arms:
Arm A: Venetoclax plus Azacitidine
Arm B: Placebo plus Azacitidine

Study Treatment: All subjects will receive venetoclax or placebo orally once daily (QD) plus Azacitidine QD beginning on Cycle 1 Day 1. Venetoclax or placebo should be taken within 30 minutes of food intake. Subjects will receive Azacitidine for 7 days from Day 1 of each cycle beginning with Cycle 1:

Cycle Length—28 Days
Venetoclax 400 milligram (mg) or Placebo Daily on Days 1-28
Azacitidine 75 mg/m² Subcutaneous (SC) or IV Daily for 7 days

Subjects will continue their treatment assignment until documented disease progression per Investigator assessment, unacceptable toxicity, withdrawal of consent, or the subject meets other protocol criteria for discontinuation (whichever occurs first). Subjects will continue to receive the assigned study treatment without cross over until treatment discontinuation. All subjects will have a final visit performed when treatment is discontinued unless the subject has withdrawn consent to participate in the study. Baseline laboratory assessments will be obtained at Cycle 1 Day 1 prior to first dose of study treatment. Patient-reported outcome (PRO) measures should be completed after a blood sample is taken to confirm the subject is able to receive study treatment at the study visit, but prior to the performance of all other non-PRO assessments and the administration of study treatment. Disease assessments by IWG criteria will be performed at end of Cycle 1 (±3 days) and every 3 cycles starting on Cycle 4 Day 1 and continuing until disease progression per the modified IWG criteria, or the subject withdraws consent. In addition to being reviewed by the Investigator and local hematopathologists, all disease assessment information will be sent to an Independent Review Committee (IRC) to provide response assessment. Interpretations from the IRC will not be shared with sites. Upon unblinding at the 75% Overall Survival Interim Analysis, disease assessments will no longer be performed by the IRC.

| | |
|---|---|
| Investigational Product: | Venetoclax, 100 mg, 50 mg and 10 mg tablet |
| Dose: | Cycle 1: Day 1: 100 mg, Day 2: 200 mg, Day 3: 400 mg, |
| | Day 4-Day 28: 400 mg |
| | Subsequent cycles: Day 1-Day 28: 400 mg QD |
| Mode of Administration: | Oral |
| Reference Therapy: | Placebo (to match venetoclax 100 mg, 50 mg, and 10 |
| | mg tablet) |
| Dose: | Not Applicable |
| Mode of Administration: | Oral |
| Reference Therapy: | Azacitidine |
| Dose: | 75 mg/m²; Daily for 7 Days of all cycles, Cycle = |
| | 28 days QD |
| Mode of Administration: | Subcutaneous (SC) or IV as indicated in local label |

Duration of Treatment: Subjects will receive venetoclax/placebo/azacitidine until documented disease progression, unacceptable toxicity or intolerance, withdrawal of consent, or the subject meets other criteria for discontinuation per study protocol (whichever occurs first).

Criteria for Evaluation:

Efficacy:

Bone marrow biopsies and aspirates must be performed at screening for all subjects. Cytogenetic and molecular profiling will be done at a local lab during screening. Samples for molecular markers and baseline disease assessment for MRD evaluation must be collected at screening for confirmation at central lab. Bone marrow aspirate and biopsy must be performed at the end of Cycle 1. For subjects with resistant disease at the end of Cycle 1 a repeat bone marrow aspirate and biopsy must be performed at the end of Cycle 2 or Cycle 3 based on the hematologic recovery to assess response. For all subjects a bone marrow aspirate and biopsy must be performed at end of Cycle 4 and every 3 cycles thereafter (±1 week).

Bone marrow will be performed until two successive samples indicate CR or CRi. For subjects with a response of CRi on two successive bone marrow samples an additional bone marrow aspirate and biopsy must be performed to confirm a CR once peripheral blood count recovery is noted. Subsequently, disease assessments are based on laboratory results and physical examination. A bone marrow aspiration and biopsy is required if relapse is suspected or at the Final Visit. All subjects will have response assessment according to the modified IWG criteria for AML. Progressive disease is defined per European LeukemiaNet recommendations. If additional treatments are needed to optimize subjects' medical care, they can be performed following institutional standards and procedures. Subject's disease assessment is based on the most recent physical examination, bone marrow results and recent hematology values. For subjects who require a delay in next cycle of study treatment for blood count recovery after a bone marrow evaluation, hematology values for up to 2 weeks or pre-dose labs from Day 1 of the next cycle can be used to determine the IWG response.

All subjects who completed at least one cycle of study treatment will be assessed by the investigators using the modified IWG criteria for AML, as described below. Subjects who have discontinued study treatment prior to completion of Cycle 1 will be deemed non-evaluable for response assessment.
CR: Absolute neutrophil count≥10³/μL, platelets≥10⁵/μL, red cell transfusion independence, and bone marrow with <5% blasts. Absence of circulating blasts and blasts with Auer rods; absence of extramedullary disease.
CRi: All of the criteria for CR except for residual neutropenia<10³/μL (1000/μL) or thrombocytopenia<10⁵/μL (100,000/μL). Red blood cell transfusion (RBC) dependence is also defined as CRi.
PR: All of the hematologic values for a CR but with a decrease of at least 50% in the percentage of blasts to 5% to 25% in the bone marrow aspirate.
MLFS: Less than 5% blasts in an aspirate sample with marrow spicules and with a count of at least 200 nucleated cells, absence of circulating blasts and extramedullary disease without peripheral blood count recovery that meet the thresholds for either CR or CRi.
RD: Failure to achieve CR, CRi, PR or MLFS; only for subjects surviving at least 7 days following completion of Cycle 1 treatment, with evidence of persistent leukemia by blood and/or bone marrow examination.
MR: Reappearance of ≥5% blasts after CR/CRi in peripheral blood or bone marrow or development of extramedullary disease.
PD:* 50% increase in marrow blasts over baseline (a minimum 15% point increase is required in cases with <30% blasts at baseline; or persistent marrow blast percentage of >70% over at least 3 months; without at least a 100% improvement in ANC to an absolute level (>0.5×10⁹/L [500/μL], and/or platelet count to >50×10⁹/L [50 000/μL] non-transfused); or
   50% increase in peripheral blasts (WBC×% blasts) to >25×10⁹/L(>25 000/μL); or
   New extramedullary disease
CR=complete remission; CRi=CR with incomplete blood count recovery; PR=partial remission; MLFS=morphologic leukemia free state; RD=resistant disease; MR=morphologic relapse
PD=Progressive disease as defined by ELN criteria.

In addition to the response assessment using the above response criteria, each subject will also be evaluated for complete remission with partial hematologic recovery rate (CRh) based on the bone marrow and hematologic parameters.

Statistical Methods:

Efficacy:

Primary and Secondary Efficacy Endpoints:

Overall Survival (OS):

Overall survival will be defined as the number of days from the date of randomization to the date of death. Subjects that have not died will be censored at the last known date to be alive.

Composite Complete Remission Rate:

The proportion of subjects with complete remission or complete remission with incomplete marrow recovery (CR+CRi) will be calculated based on current IWG criteria for AML. Subjects who are randomized but have no IWG disease assessment will be considered as non-responders for CR+CRi rate.

Complete Remission (CR) Rate:

The proportion of subjects with complete remission (CR) will be calculated based on current IWG criteria for AML. Subjects who are randomized but have no IWG disease assessment will be considered as non-responders for CR rate.

CR+CRh Rate:

The proportion of subjects with complete remission or complete remission with incomplete marrow recovery (CR+CRh) will be calculated. Subjects who are randomized but have no post-baseline disease assessment will be considered as non-responders for CR+CRh rate.

Composite Complete Remission Rate by the Initiation of Cycle 2:

The proportion of subjects with complete remission or complete remission with incomplete marrow recovery (CR+CRi) by the initiation of Cycle 2 will be calculated based on the modified IWG criteria for AML. Subjects who are randomized but have no IWG disease assessment by the initiation of Cycle 2 will be considered as non-responders.

Post Baseline RBC Transfusion Independence Rate:

Post baseline RBC transfusion independence rate will be calculated as the portion of subjects who achieved RBC transfusion independence post baseline. The RBC transfusion independence is defined as a period of at least 56 days with no RBC transfusion between the first dose of study drug and the last dose of study drug+30 days. All randomized subjects will be included to estimate the post-baseline transfusion independence rates.

The Rate of Conversion (RBC):

The rate of conversion will be calculated as proportion of subjects being post-baseline transfusion independent from baseline RBC transfusion dependence.

The Rate of Conversion (Platelets):

The rate of conversion will be calculated as proportion of subjects being post-baseline Platelets transfusion independent from baseline platelets transfusion dependence.

MRD Response Rate:

MRD response will be defined using a threshold of less than 0.1% of residual blasts per leukocytes as measured in bone marrow. Additional thresholds may also be explored and correlated with efficacy outcomes. Subjects who are randomized but have no MRD assessment will be considered as non-responders for the calculation of MRD response rate. The proportion of subjects (CR+CRi, or CR+CRh) achieving an MRD response will be calculated.

Event-Free Survival (EFS):

EFS will be defined as the number of days from randomization to the date of progressive disease, relapse from CR or CRi, treatment failure or death from any cause. If a specified event does not occur, subjects will be censored at the date of last disease assessment. Data for subjects without any disease assessments performed after randomization will be censored at the date of randomization.

Reporting of Results

All dosed subjects will be assessed for response to treatment based on the published guidelines. Assessments will be performed at the end of Cycle 1 and every 3 cycles thereafter for response assessment. Subject will be assigned to one or more of the following categories by the investigators: (1) complete remission; (2) complete remission with incomplete blood count recovery; (3) partial remission; (4) morphologic leukemia free state; (5) resistant disease; (6) progressive disease; (7) indeterminate (not assessable, insufficient data); and (8) morphologic relapse. MRD status if performed at investigator site for subjects who achieve CR or CRi: (1) minimal residual disease negative; (2) minimal residual disease positive.

Management of Cytopenia:

If a subject achieves CRi or has a morphologic leukemia free bone marrow (MLFS) (i.e., bone marrow blasts<5%) after completion of Cycle 1, venetoclax/placebo should be interrupted to allow for ANC recovery from Day 29 until ANC>500/μL or up to 14 days. Cycle 2 administration of azacitidine will also be delayed until ANC≥500/μL. Both venetoclax/placebo and azacitidine will resume on the same day after the interruption. If a subject presents with new onset Grade 4 neutropenia for more than 1 week during subsequent cycles, unless it is thought to be due to the underlying disease, venetoclax/placebo dosing should be interrupted until ANC is ≥500/μL. After Cycle 3, for subjects in CR/CRi who required interruption or delay of study drug administration for cytopenia (neutropenia [≤500/μL] or thrombocytopenia [≤50×10³/μL]) venetoclax/placebo should be administered for 21 days out of 28 days during each of the subsequent cycles. Treatment cycle should also be delayed to allow for count recovery until ANC≥500/μL and/or platelet count≥50×10³/μL or for up to 14 days whichever occurs earlier.

Subjects with ANC of 1000/μL and platelet count≥100×10³/μL at end of Cycle 1 may proceed to Cycle 2 before the results of the bone marrow aspirate and biopsy preformed at end of Cycle 1 become available.

Subjects with resistant disease after Cycle 1 should receive subsequent cycles of study treatment with no dose interruption/delay until a repeat bone marrow assessment demonstrates CRi or MLFS. Once this response is achieved, dose interruptions and reduction in duration of venetoclax administration for neutropenia should be implemented as described above beginning from the cycle where a CRi or MLFS is demonstrated. If hematologic recovery (ANC or platelets) is achieved within 14 days after completion of the cycle, the duration of venetoclax is reduced to 21 days of the subsequent 28-day cycle. During subsequent cycles, if hematologic recovery with more than 25% increase above the nadir is not seen by Day 28, reassess counts every 7 days. If a 25% increase has not been achieved within 14 days after the completion of a cycle, based on the bone marrow biopsy cellularity azacitidine dose adjustment can be made according to Table 1, below:

TABLE 1
Azacitidine dose modification

| | % Dose in the Next Cycle | |
|---|---|---|
| Bone Marrow | if Recovery is Not Achieved Within 14 Days | |
| Cellularity | Recovery ≤21 Days | Recovery >21 Days |
| 15-50% | 100% | 50% |
| <15% | 100% | 33% |

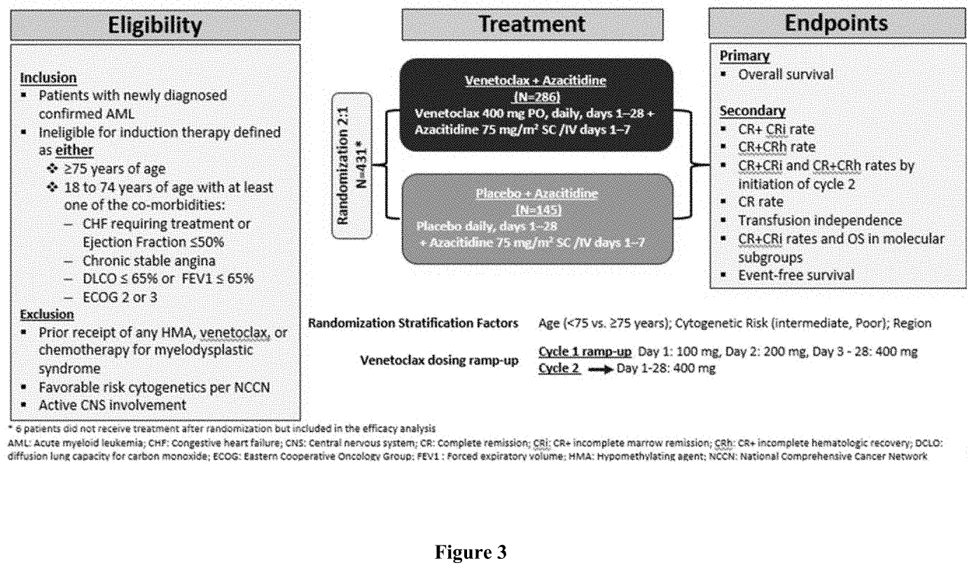

An exemplary dosing modification for venetoclax and azacitidine to manage cytopenia is shown in FIG. 3.

Endpoints:

Primary endpoints are CR+CRi rate and overall survival (OS).

Secondary endpoints are CR+CRi rate, CR+CRh rate, CR+CRi at initiation of Cycle 2, CR rate, post-baseline transfusion independence rates for RBC and/or platelets, the rates of conversion from baseline transfusion dependence to post-baseline transfusion independence post-baseline.

CRh (Complete remission with partial hematologic recovery) is a derived response based on bone marrow blast and hematology lab values. A response of CRh is achieved when the following criteria are met: (1) Bone marrow with <5% blasts; (2) peripheral blood neutrophil count of ≥0.5×10³/μL; an (3) peripheral blood platelet count of ≥0.5×10⁵/μL.

### 7.2 Example 2: A Double-Blind, Placebo-Controlled, Multicenter Phase 3 Study of Venetoclax and Azacitidine in Patients with Acute Myeloid Leukemia

Background: Patients with acute myeloid leukemia (AML) who are older or ineligible for intensive induction chemotherapy have limited treatment options and poor survival. In this study, venetoclax (Ven)+azacitidine (Aza) improved overall survival and response rates compared with placebo (Pbo)+Aza in older or unfit patients with newly diagnosed AML. While cytopenia is common in AML, Ven+Aza was associated with hematologic adverse events in 83% of patients in this study (versus 69% in the Pbo+Aza arm). In this example, the frequency and management of cytopenia are analyzed in patients achieving a best response of complete remission (CR) or CR with partial hematologic recovery (CRh).

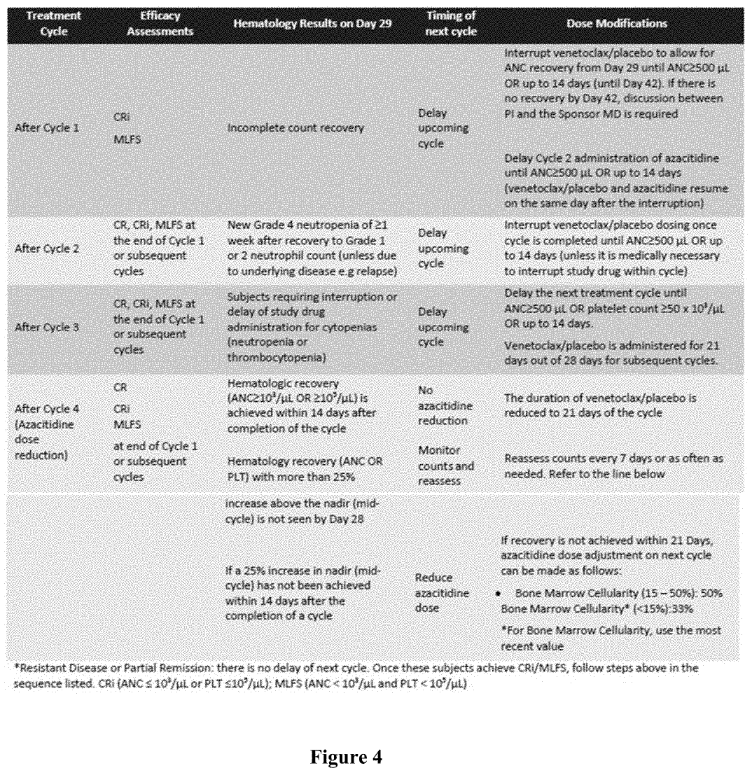

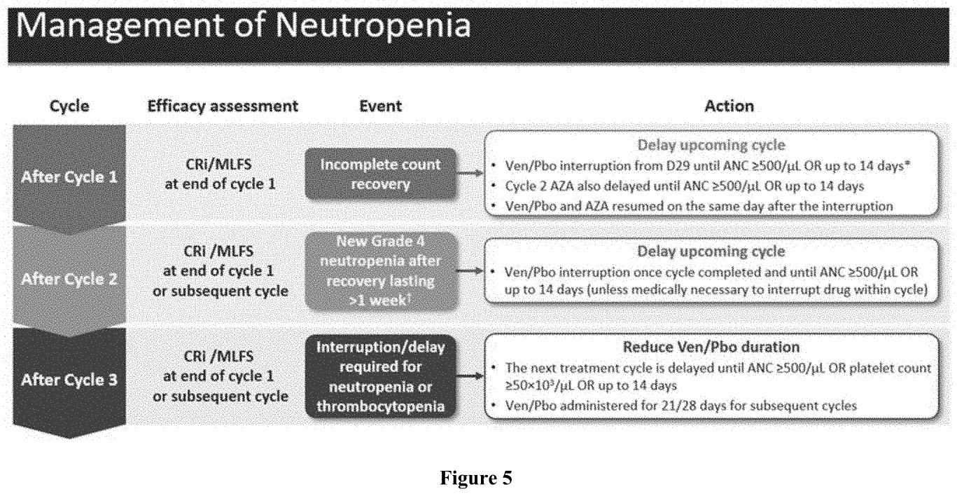

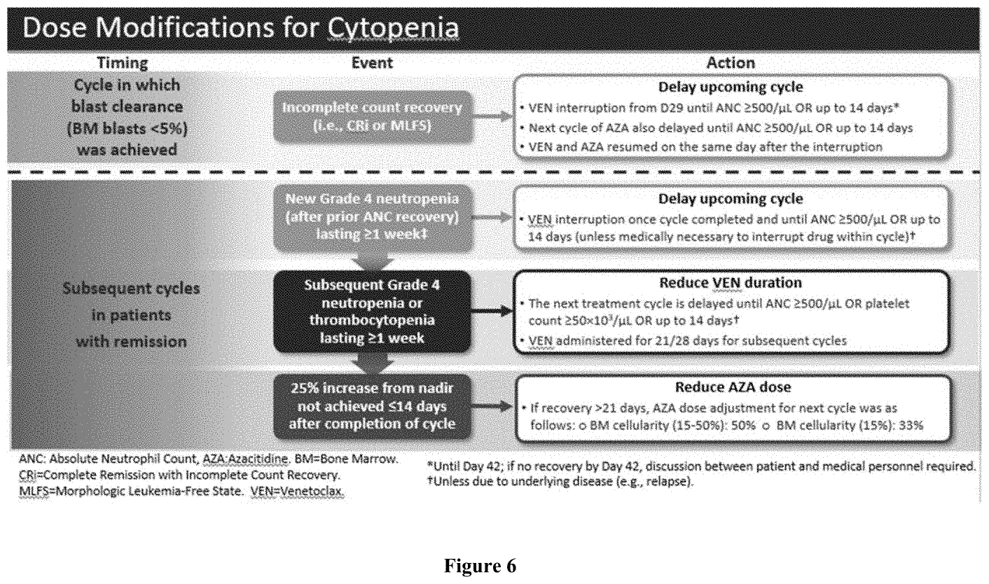

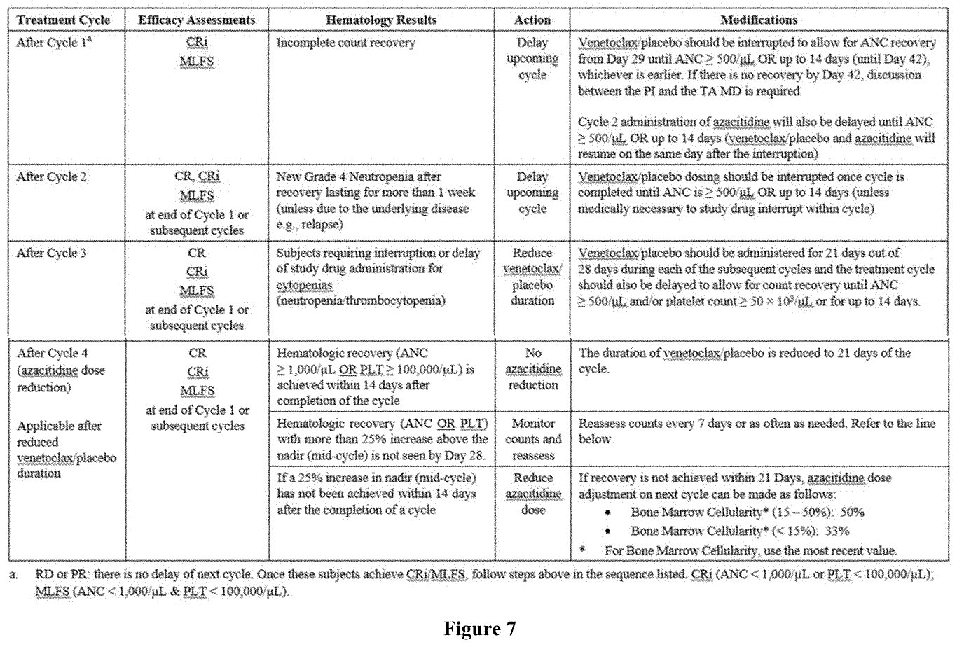

Methods: This double-blind, Pbo-controlled, multicenter Phase 3 study enrolled patients with newly diagnosed AML who were ineligible for intensive chemotherapy due to age≥75 years or comorbidities. Patients were randomized 2:1 to receive 75 mg/m²Aza (Days 1-7 of each 28-day cycle) plus either daily 400 mg Ven (Ven+Aza) or Pbo (Pbo+Aza). FIG. 3 shows an exemplary study design. Disease response was assessed via bone marrow aspirate and biopsy at the end of Cycle 1 and at least every 3 cycles thereafter. After patients achieved blast clearance (bone marrow blasts<5%), various dosing modifications were implemented to manage cytopenia (see Table 2 below; see also FIGS. 4-7). Cytopenia, defined here as Grade 4 neutropenia (absolute neutrophil count<500/μL) or Grade 4 thrombocytopenia (platelet count<25×10³/μL) lasting>7 days, was assessed using laboratory data.

TABLE 2
Dosing modifications to manage cytopenia

| Timing | Event | Action |
|---|---|---|
| Cycle in which | Incomplete count | Delay upcoming cycle |
| blast clearance | recovery (ie, CRi or | Ven/Pbo interruption from Day 29 until ANC ≥500/μL or |
| (BM blasts | MLFS) | up to 14 days^{a,b} |
| <5%) was | | Next cycle of Aza also delayed until ANC ≥500/μL or up |
| achieved | | to 14 days^{b} |
| | | Ven/Pbo and Aza resumed on the same day after the |
| | | interruption |
| Subsequent cycles in patients with remission | 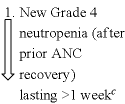 | Delay upcoming cycle Ven/Pbo interruption once cycle completed and until ANC ≥500/μL or up to 14 days (unless medically necessary to interrupt drug within cycle) |
| | 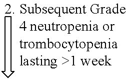 | Reduce Ven/Pbo duration The next treatment cycle is delayed until ANC ≥500/μL or platelet count ≥50 × 10³/μL or up to 14 days Ven/Pbo administered for 21/28 days for subsequent cycles |
| | 3. 25% increase from | Reduce Aza dose |
| | nadir not achieved | If recovery >21 days, Aza dose adjustment for next cycle |
| | ≤14 days after | was as follows: |
| | completion of cycle | BM cellularity (15-50%): 50% |
| | | BM cellularity (<15%): 33% |

^{a}Until Day 42; if no recovery by Day 42, a discussion between the investigator and the Abb Vie MD was required.
^{b}Granulocyte-colony stimulating factor support was used per institutional practice.
^{c}Unless due to underlying disease (eg, relapse).
ANC, absolute neutrophil count; Aza, azacitidine; BM, bone marrow; CRi, complete remission with incomplete count recovery; MLFS, morphologic leukemia-free state; Pbo, placebo; Ven, venetoclax.

Results: In total, 186/283 (66%) Ven+Aza-treated patients and 33/144 (23%) Pbo+Aza-treated patients achieved a best response of CR or CRh (CR/CRh). Of patients with a best response of CR/CRh in the Ven+Aza arm, 77% achieved blast clearance by the end of Cycle 1, 88% by the end of Cycle 2, 92% by the end of Cycle 3, and 98% by the end of Cycle 4. Of patients with a best response of CR/CRh in the Pbo+Aza arm, 33% achieved blast clearance by the end of Cycle 1, 55% by the end of Cycle 2, 76% by the end of Cycle 3, and 91% by the end of Cycle 4. After achieving blast clearance, 75% and 67% of patients in the Ven+Aza and Pbo+Aza arms, respectively, had a delay of the next cycle, with a median duration per cycle delay post-blast clearance (range) of 9.0 (1-39) and 5.5 (1-21) days. Among CR/CRh patients, more patients receiving Ven+Aza versus Pbo+Aza had post-remission cytopenia (87% vs 45%; see Table 3 below). A similar percentage of CR/CRh patients in both arms (Ven+Aza: 26%; Pbo+Aza: 24%) had in-cycle dose interruptions (ie, days without Ven/Pbo exposure between a cycle's first and last Ven/Pbo dose), with a median duration (range) of 2.0 days (1-20) for Ven+Aza and 1.0 (1-13) for Pbo+Aza. A greater proportion of CR/CRh patients experienced post-remission cycle delays due to cytopenia in the Ven+Aza arm (77%) than in the Pbo+Arm (30%). Furthermore, a higher percentage of CR/CRh patients had post-remission cycles with a reduction in Ven/Pbo dosing days and/or cycle delays totaling ≥7 days due to cytopenia in the Ven+Aza arm (75%) than in the Pbo+Aza arm (27%). Among these patients, the median percentage of days without Ven/Pbo administration while in remission (out of the total number of days in remission) was 18% (range: 1-69) in the Ven+Aza arm and 13% (3-44) in the Pbo+Aza arm. Ultimately, 129 CR/CRh patients (69%) in the Ven+Aza arm and 10 (30%) in the Pbo+Aza arm received ≤21-day Ven/Pbo dosing post-remission, with a median time from remission to first ≤21-day cycle (range) of 92.0 days (1-480) for Ven+Aza and 74.0 days (6-405) for Pbo+Aza.

TABLE 3
Cytopenia and dosing modifications among patients who achieved a best response
of CR/CRh

| | Ven + Aza | Pbo + Aza |
|---|---|---|
| | (n = 186) | (n = 33) |
| Number of responders with post-remission Grade 4 | | |
| cytopenia episodes lasting ≥1 week, n (%) | | |
| 0 episodes | 24 (13) | 18 (55) |
| 1 episode | 36 (19) | 8 (24) |
| ≥2 episodes | 126 (68) | 7 (21) |
| Number of responders with in-cycle dose interruptions for | | |
| any reason, n (%) | | |
| 0 cycles | 138 (74) | 25 (76) |
| 1 cycle | 33 (18) | 5 (15) |
| ≥2 cycles | 15 (8) | 3 (9) |
| Number of responders with post-remission cycle delays due | | |
| to cytopenia | | |
| 0 cycle delays, n (%) | 42 (23) | 23 (70) |
| 1 cycle delay, n (%) | 36 (19) | 4 (12) |
| ≥2 cycle delays, n (%) | 108 (58) | 6 (18) |
| Median duration per cycle delay (range), days | 14.0 (1-129) | 11.0 (3-63) |
| Number of responders who had post-remission cycles with a | | |
| reduction of Ven/Pbo dosing days and/or cycle delay | | |
| totaling ≥7 days due to cytopenia | | |
| 0 cycles, n (%) | 47 (25) | 24 (73) |
| 1 cycle, n (%) | 30 (16) | 3 (9) |
| ≥2 cycles, n (%) | 109 (59) | 6 (18) |
| Median number of cycles (range) | 2.0 (0-15) | 0 (0-7) |

Aza, azacitidine;
CR, complete remission;
CRh, complete remission with partial hematologic recovery;
Pbo, placebo;
Ven, venetoclax.

Conclusion: In this study of older or unfit patients with newly diagnosed AML, the majority of responding patients in the Ven+Aza arm required dosing modifications to manage cytopenia, of which delays between cycles or within cycle reductions of Ven dosing days were most common. Post-remission cytopenia and dosing modifications were more frequent with Ven+Aza versus Pbo+Aza treatment.

### 7.3 Example 3: Measurable Residual Disease Response in Acute Myeloid Leukemia Treated with Venetoclax and Azacitidine

Background: Rates of composite complete remission (CRc; complete remission [CR]+CR with incomplete hematologic recovery [CRi]) and measurable residual disease response (MRD<10⁻³) were higher in patients (patients) treated with venetoclax (Ven)+azacitidine (Aza) compared to Aza alone (23.4%/7.6%, p<0.001). In this example, the outcomes of patients treated with Ven+Aza who achieved both CRc and MRD<10⁻³ in Example 2 was explored.

Methods: Enrolled patients were ≥18 years and unfit for intensive chemotherapy. Patients received Ven 400 mg orally; days 1-28 and Aza 75 mg/m²; days 1-7/28-day cycle. Bone marrow aspirate samples for multiparametric flow cytometry assessments by integrated leukemia-associated immunophenotypes and different than normal procedures were collected for central analysis (Covance Central Laboratory Services) at baseline, end of cycle 1, and every 3 cycles thereafter. Assessments were performed independent of disease responses. MRD response was defined as <1 residual blast/1000 leukocytes (<10⁻³). CRc, DoR, OS, and EFS were assessed. Disease assessments were per modified International Working Group response criteria for AML.

Results: 211/286 (74%) patients treated with Ven+Aza with at least one valid post-baseline MRD assessment were considered MRD evaluable; 78/211 (37%) achieved MRD<10⁻³ and 133/211 (63%) had MRD≥10⁻³. Median age (MRD<10⁻³/MRD≥10⁻³) was 76 (range: 49-89)/77 (58-91) years.

Patients (MRD<10⁻³/MRD≥10⁻³) received median of 14.5 (range: 1-28)/7.0 (1-30) cycles of Ven+Aza. At a median follow-up of 22.0 (range: 20.1-23.0)/20.8 (19.8-22.3) months (mos), CRc+MRD<10⁻³/MRD≥10⁻³ was achieved by 67 (86%)/97 (73%); 20/67 (30%) achieved CRc+MRD<10⁻³ by end of cycle 1.

Median DoR, OS, and EFS were not reached in patients with CRc+MRD<10⁻³ response (see Table 4 below). The 12-mo estimates for DoR, OS, and EFS for patients with CRc+MRD<10⁻³ response were 81.2%, 94.0%, and 83.2%, respectively.

Adverse events≥grade 3 (MRD<10⁻³/MRD≥10⁻³) were febrile neutropenia (50%/43%), neutropenia (50%/35%), and thrombocytopenia (44%/44%), similar to the overall population.

TABLE 4
DoR, OS, and EFS in patients with composite complete response
treated with venetoclax and azacitidine

| | 12-mos estimate | | Median months | |
|---|---|---|---|---|
| | % (95% CI) | | (95% CI) | |
| | MRD <10⁻³ | MRD ≥10⁻³ | MRD <10⁻³ | MRD ≥10⁻³ |
| | n = 67 | n = 97 | n = 67 | n = 97 |
| Duration of | 81.2 (69.3, 88.9) | 46.6 (35.6, 56.8) | NR (19.3, NR) | 9.7 (8.0. 15.8) |
| response | | | | |
| Overall | 94.0 (84.7, 97.7) | 67.9 (57.6, 76.2) | NR (24.4, NR) | 18.7 (12.9, NR) |
| survival | | | | |
| Event-free | 83.2 (71.6, 90.3) | 45.4 (35.2, 55.0) | NR (19.7, NR) | 10.6 (9.0, 13.9) |
| survival | | | | |

CI: Confidence interval;
MRD: measurable residual disease;
NR: not reached

Conclusion: Patients with best response of CRc who achieved MRD<10⁻³ response with Ven+Aza treatment had longer DoR, OS, and EFS than patients who were CRc and MRD positive.

### 7.4 Example 4: Overall Survival in Patients with CR/CRh with Post-Remission Cytopenia

Background: This example explores the relationship between venetoclax exposure and post-remission cytopenia, as defined by Grade 4 neutropenia or thrombocytopenia lasting≥7 days, in patients who achieved complete remission (CR) or complete remission with partial hematological recovery (CRh) in the study of Example 2. This example also explores the relationship between venetoclax exposure and overall survival in subgroups of patients with post-remission cytopenia.

Methods: Data from 185 patients treated with VEN+AZA and 33 patients treated with PBO+AZA who achieved CR or CRh (blast clearance with neutrophil count>0.5×10³/μL and platelet count>0.5×10⁵/μL) were included in the analysis. Venetoclax exposure was calculated as the average plasma concentration (C_{avg}) from blast clearance to the event of interest, accounting for dose modifications such as interruptions and reductions. Logistic regression and Cox proportional-hazards models were used to characterize the relationships between venetoclax exposure and frequency or timing of post-remission cytopenia. Cox proportional-hazards models were used to characterize the relationships between venetoclax exposure and overall survival.

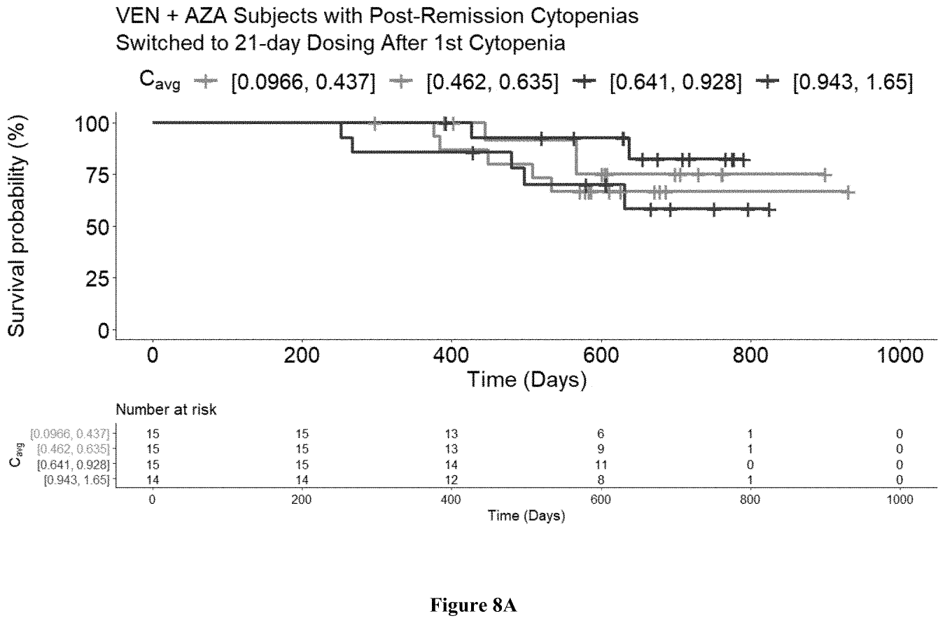

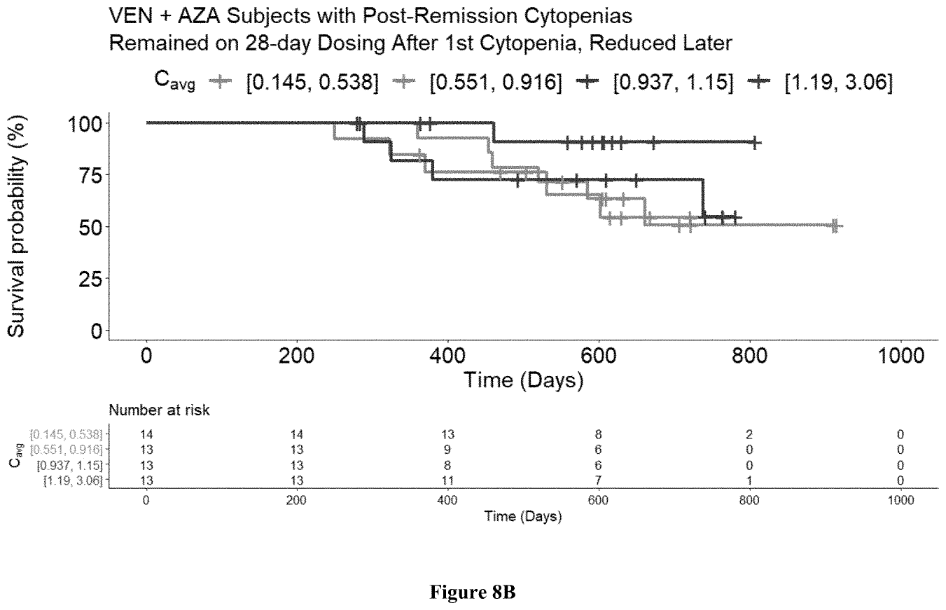

Results: There is no apparent relationship between venetoclax exposure and overall survival in subgroups of patients who switched to 21-day dosing following their first post-remission cytopenia (see FIG. 8A) or remained on 28-day dosing and reduced venetoclax dosing duration in later cycles (see FIG. 8B). These results indicate that lower exposures associated with venetoclax dose reductions to manage a cytopenia in patients who achieved CR/CRh appear to have overall survival similar to AML, patients that did not experience a cytopenia.

Conclusion: Lower exposures associated with venetoclax dose reductions to manage cytopenia in patients who achieved CR/CRh does not appear to diminish overall survival, confirming that the methods described herein successfully manage cytopenia in patients on combination therapies of venetoclax and azacitidine to treat AML.

The embodiments described herein are intended to be merely exemplary, and those skilled in the art will recognize, or will be able to ascertain using no more than routine experimentation, numerous equivalents of specific compounds, materials, and procedures. All such equivalents are considered to be within the scope of the disclosure.

All of the patents, patent applications and publications referred to herein are incorporated by reference herein in their entireties. Citation or identification of any reference in this application is not an admission that such reference is available as prior art to this application. The full scope of the disclosure is better understood with reference to the appended claims.

1-4. (canceled)

5. A method of managing cytopenia in a patient with neutropenia and/or thrombocytopenia when on a combination therapy of venetoclax and azacitidine to treat acute myeloid leukemia (AML), the method comprising:
a) administering venetoclax and azacitidine to a patient in need thereof for one or more 28-day cycles of a multiple 28-day cycle dosing regimen, wherein each 28-day cycle of the one or more 28-day cycles comprises orally administering 400 mg venetoclax on days 1-28 of the 28-day cycle, and administering azacitidine 75 mg/m² intravenously or subcutaneously on days 1-7 of the 28-day cycle; and
b) interrupting the administration of venetoclax and azacitidine to the patient after a first 28-day cycle of the one or more 28-day cycles in step (a), upon the patient having:
(i) an occurrence of neutropenia, until the patient has absolute neutrophil count (“ANC”) greater than or equal to 500 cells/μL within 14 days from the day of the interruption, and/or
(ii) an occurrence of thrombocytopenia, until the patient has a platelet count greater than or equal to 50,000 cells/μL within 14 days from the day of the interruption; and
resuming the administration of venetoclax and azacitidine for the next 28-day cycle of the one or more 28-day cycles in step (a), wherein the resumed administration of venetoclax and azacitidine starts on the same day.

6. The method of claim 5, further comprising:
c) interrupting the administration of venetoclax to the patient after a second 28-day cycle of the resumed one or more 28-day cycles in step (a), while azacitidine continues to be administered to the patient as in step (a), upon the patient having:
(i) an occurrence or reoccurrence of neutropenia, until the patient has absolute neutrophil count (“ANC”) greater than or equal to 500 cells/μL within 14 days from the day of the interruption, and/or
(ii) an occurrence or reoccurrence of thrombocytopenia, until the patient has a platelet count greater than or equal to 50,000 cells/μL within 14 days from the day of the interruption; and
resuming the administration of venetoclax for the next 28-day cycle of the one or more 28-day cycles in step (a).

7. The method of claim 6, further comprising:
d) interrupting the administration of venetoclax to the patient after a third 28-day cycle of the resumed one or more 28-day cycles in step (a), while azacitidine continues to be administered to the patient as in step (a), upon the patient having:
(i) an occurrence or reoccurrence of neutropenia, until the patient has an ANC greater than or equal to 500 cells/μL within 14 days from the day of the interruption, and/or
(ii) an occurrence or reoccurrence of thrombocytopenia, until the patient has a platelet count greater than or equal to 50,000 cells/μL within 14 days from the day of the interruption; and
resuming the administration of venetoclax only on days 1-21 of the next 28-day cycle of the one or more 28-day cycles in step (a).

8. The method of claim 7, further comprising:
e) reducing the dose of azacitidine intravenously or subcutaneously administered to the patient on days 1-7 of the next 28-day cycle after the fourth 28-day cycle in step (a), if the patient in step (c) and/or step (d) does not have an ANC greater than or equal to 500 cells/μL, or if the patient in step (c) and/or step (d) does not have a platelet count greater than or equal to 50,000 cells/μL, within 14 days from the day of the interruption, and the patient within 21 days from the day of the interruption recovers to having an ANC greater than or equal to 500 cells/μL and having a platelet count greater than or equal to 50,000 cells/μL, and
resuming the 28-day cycles in step (a) wherein the administered dose of azacitidine is reduced to (1) 37.5 mg/m² azacitidine when the recovered patient has a bone marrow cellularity level of between 15-50%, or (2) 25 mg/m² azacitidine when the recovered patient has a bone marrow cellularity of less than 15%.

9. The method of claim 5, wherein if the patient in step (b) does not have an ANC greater than or equal to 500 cells/L within 14 days from the day of the interruption, excluding the patient from the method of treatment.

10. The method of claim 5, wherein the patient has achieved complete recovery (CR), complete remission with incomplete count recovery (CRi) or a morphologic leukemia-free state (MLFS) prior to step (b).

11. The method of claim 7, wherein the occurrence or reoccurrence of neutropenia in step (c) and step (d) lasts for at least one week and is not due to a relapse of AML.

12. The method of claim 7, wherein each occurrence or reoccurrence of neutropenia in step (b), (c) and (d) includes the patient having an ANC that is less than 1,000 cells/μL, and each occurrence or reoccurrence of thrombocytopenia in step (b), (c) and (d) includes the patient having a platelet count that is less than 100,000 cells/μL.

13. The method of claim 7, wherein the patient has a bone marrow blast level of less than 5% after venetoclax and azacitidine are administered to the patient in step (a) and prior to the interrupting in step (b), (c) and (d).

14. The method of claim 5, wherein, optionally, on days 1-3 of the first 28-day cycle of the one or more 28-day cycles step (a), the amount of venetoclax orally administered to the patient is 100 mg on day 1, 200 mg on day 2, and 400 mg on day 3.

15. The method of claim 5, wherein the neutropenia is a Grade 4 neutropenia.

16. (canceled)

17. A method of managing cytopenia in a patient with neutropenia and/or thrombocytopenia when on a combination therapy of venetoclax and azacitidine to treat acute myeloid leukemia (AML), the method comprising:
a) administering venetoclax and azacitidine to a patient in need thereof for one or more 28-day cycles of a multiple 28-day cycle dosing regimen, wherein each 28-day cycle of the one or more 28-day cycles comprises orally administering 400 mg venetoclax on days 1-28 of the 28-day cycle, and administering azacitidine 75 mg/m² intravenously or subcutaneously on days 1-7 of the 28-day cycle;
b) interrupting the administration of venetoclax and azacitidine to the patient after a first 28-day cycle of the one or more 28-day cycles in step (a), upon the patient having:
(i) an occurrence of neutropenia, until the patient has absolute neutrophil count (“ANC”) greater than or equal to 500 cells/μL within 14 days from the day of the interruption, and/or
(ii) an occurrence of thrombocytopenia, until the patient has a platelet count greater than or equal to 50,000 cells/μL within 14 days from the day of the interruption; and
resuming the administration of venetoclax and azacitidine for the next 28-day cycle of the one or more 28-day cycles in step (a), wherein the resumed administration of venetoclax and azacitidine starts on the same day;
c) interrupting the administration of venetoclax to the patient after a second 28-day cycle of the resumed one or more 28-day cycles in step (a), while azacitidine continues to be administered to the patient as in step (a), upon the patient having:
(i) an occurrence or reoccurrence of neutropenia, until the patient has absolute neutrophil count (“ANC”) greater than or equal to 500 cells/μl, within 14 days from the day of the interruption, and/or
(ii) an occurrence or reoccurrence of thrombocytopenia, until the patient has a platelet count greater than or equal to 50,000 cells/μl, within 14 days from the day of the interruption; and
resuming the administration of venetoclax for the next 28-day cycle of the one or more 28-day cycles in step (a);
d) interrupting the administration of venetoclax to the patient after a third 28-day cycle of the resumed one or more 28-day cycles in step (a), while azacitidine continues to be administered to the patient as in step (a), upon the patient having:
(i) a reoccurrence or reoccurrence of neutropenia, until the patient has an ANC greater than or equal to 500 cells/μl, within 14 days from the day of the interruption, and/or
(ii) an occurrence or reoccurrence of thrombocytopenia, until the patient has a platelet count greater than or equal to 50,000 cells/μl, within 14 days from the day of the interruption; and
resuming the administration of venetoclax only on days 1-21 of the next 28-day cycle of the one or more 28-day cycles in step (a); and
e) reducing the dose of azacitidine intravenously or subcutaneously administered to the patient on days 1-7 of the next 28-day cycle after the fourth 28-day cycle in step (a), if the patient in step (c) and/or step (d) does not have an ANC greater than or equal to 500 cells/μL, or if the patient in step (c) and/or step (d) does not have a platelet count greater than or equal to 50,000 cells/μL, within 14 days from the day of the interruption, and the patient within 21 days from the day of the interruption recovers to having an ANC greater than or equal to 500 cells/μL and having a platelet count greater than or equal to 50,000 cells/μL, and
resuming the 28-day cycles in step (a) wherein the administered dose of azacitidine is reduced to (1) 37.5 mg/m² azacitidine when the recovered patient has a bone marrow cellularity level of between 15-50%, or (2) 25 mg/m² azacitidine when the recovered patient has a bone marrow cellularity of less than 15%;
wherein:
1) the occurrence or reoccurrence of neutropenia in step (c) and/or step (d) lasts for at least one week and is not due to a relapse of AML;
2) each occurrence and reoccurrence of neutropenia includes the patient having an ANC that is less than 1,000 cells/μL, and each occurrence or reoccurrence of thrombocytopenia includes the patient having a platelet count that is less than 100,000 cells/μL; and
3) the patient has a bone marrow blast level of less than 5% after venetoclax and azacitidine are administered to the patient in step (a) and prior to interrupting step (b), (c) and (d).

18. The method of claim 17, wherein, the neutropenia is a Grade 4 neutropenia.

19.-20. (canceled)

21. The method of claim 8, wherein the neutropenia is a Grade 4 neutropenia.

22. The method of claim 17, wherein on days 1-3 of the first 28-day cycle of the one or more 28-day cycles step (a), the amount of venetoclax orally administered to the patient is 100 mg on day 1, 200 mg on day 2, and 400 mg on day 3.
